(19) United States
(12) Patent Application Publication
(10) Pub. No.: US 2008/0096807 A1
(43) Pub. Date: Apr. 24, 2008

(54) Cyclic vasoactive intestinal peptide receptor-2 agonists

(76) Inventors: David Robert Bolin, Montclair, NJ (US); Wajiha Adnan Khan, East Hanover, NJ (US); Hanspeter Michel, Bloomfield, NJ (US)

Correspondence Address: HOFFMANN-LA ROCHE INC.;PATENT LAW DEPARTMENT, 340 KINGSLAND STREET, NUTLEY, NJ, 07110, US

(21) Appl. No.: 11/825,105

(22) Filed: Jul. 3, 2007

Related U.S. Application Data
(60) Provisional application No. 60/818,805, filed on Jul. 6, 2006.

(51) Int. Cl. A61K 38/16; A61P 11/00; C07K 14/00

(52) U.S. Cl. 514/11; 530/317

(57) ABSTRACT

The present invention comprises a VPAC-2 receptor agonist of the formula (I): 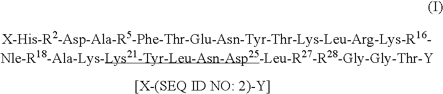 or a pharmaceutically acceptable salt thereof. Underlined residues indicate a side-chain to side-chain covalent linkage of the first and last amino acids within the segment. The present invention also encompasses pharmaceutical compositions containing such agonists, and the use of such agonists for the treatment of pulmonary diseases including COPD.

### PRIORITY TO RELATED APPLICATIONS

This application claims priority under 35 U.S.C. §119(e) of provisional application Ser. No. 60/818,805, filed Jul. 6, 2006.

### BACKGROUND OF THE INVENTION

Vasoactive intestinal peptide (VIP) was first discovered, isolated and purified from porcine intestine. [U.S. Pat. No. 3,879,371]. The peptide has twenty-eight (28) amino acids and bears extensive homology to secretin and glucagon. [Carlquist et al., Horm. Metab. Res., 14, 28-29 (1982)]. The amino acid sequence of VIP is as follows:

(SEQ ID NO:1)

His-Ser-Asp-Ala-Val-Phe-Thr-Asp-Asn-Tyr-Thr-Arg-
Leu-Arg-Lys-Gln-Met-Ala-Val-Lys-Lys-Tyr-Leu-Asn-
Ser-Ile-Leu-Asn

VIP is known to exhibit a wide range of biological activities throughout the gastrointestinal tract and circulatory system. In light of its similarity to gastrointestinal hormones, VIP has been found to stimulate pancreatic and biliary secretion, hepatic glycogenolysis, glucagon and insulin secretion and to activate pancreatic bicarbonate release. [Kerrins, C. and Said, S. L, Proc. Soc. Exp. Biol. Med., 142, 1014-1017 (1972); Domschke, S. et al., Gastroenterology, 73, 478-480 (1977)].

Two types of VIP receptors are known and have been cloned from human, rat, mouse, chicken, fish and frog. They are currently identified as VPAC1 and VPAC2 and respond to native VIP with comparable affinity. VPAC2 receptor mRNA is found in the human respiratory tract including tracheal and bronchial epithelium, glandular and immune cells, alveolar walls and macrophages. [Groneberg et al, Lab. Invest., 81, 749-755 (2001) and Laburthe et al., Receptors and Channels, 8, 137-153 (2002)].

Neurons containing VIP have been localized by immunoassay in cells of the endocrine and exocrine systems, intestine and smooth muscle. [Polak, J. M. et al. Gut, 15, 720-724 (1974)]. VIP has been found to be a neuroeffector causing the release of several hormones including prolactin [Frawley, L. S—, et al., Neuroendocrinology, 33, 79-83 (1981)], thyroxine [Ahren, B., et al. Nature, 287, 343-345 (1980)], and insulin and glucagon [Schebalin, M., et al., Am. J. Physiology E., 232. 197-200 (1977)]. VIP has also been found to stimulate renin release from the kidney in vivo and in vitro. [Porter, J. P., et al., Neuroendocrinology, 36, 404-408 (1983)]. VIP has been found to be present in nerves and nerve terminals in the airways of various animal species and man. [Dey, R. D., and Said, S. I., Fed. Proc., 39, 1062 (1980); Said, S. L, et al., Ann. N.Y. Acad. Sci., 221, 103-114, (1974)]. VIP's cardiovascular and bronchopulmonary effects are of interest as VIP has been found to be a powerful vasodilator and potent smooth muscle relaxant, acting on peripheral, pulmonary, and coronary vascular beds. [Said, S. L, et al., Clin. Res., 20, 29 (1972)]. VIP has been found to have a vasodilatory effect on cerebral blood vessels. [Lee, T. J. and Berszin, I., Science, 224, 898-900 (1984)]. In vitro studies have demonstrated that vasoactive intestinal peptide, applied exogenously to cerebral arteries, induced vasodilation, suggesting VIP as a possible transmitter for cerebral vasodilation. [Lee, T. and Saito, A., Science, 224, 898-901 (1984)]. In the eye, VIP has also been shown to be a potent vasodilator [Nilsson. S. F. E. and Bill. A., Acta Physiol. Scand., 121. 385-392 (1984)].

VIP may have regulatory effects on the immune system. O'Dorisio et al. have shown that VIP can modulate the proliferation and migration of lymphocytes. [J. Immunol., 135, 792s-796s (1985)]. Native VIP has been shown to inhibit IL-12 production in LPS-stimulated macrophages with effects on IFNγ synthesis [Delgado et al, J. Neuroimmunol., 96, 167-181 (1999)] VIP inhibits TGF-β1 production in murine macrophages and inhibits IL-8 production in human monocytes through NFκB. [Sun et al, J. Neuroimmunol., 107, 88-99 (2000) and Delgado and Ganea, Biochem. Biophys. Res. Commun., 302, 275-283 (2003)]

Since VIP has been found to relax smooth muscle and it is normally present in airway tissues, as noted above, it has been hypothesized that VIP may be an endogenous mediator of bronchial smooth muscle relaxation. [Dey, R. D. and Said, S. L., Fed. Proc., 39, 1962 (1980)]. It has been shown that tissues from asthmatic patients contain no immunoreactive VIP, as compared to tissue from normal patients. This may be indicative of a loss of VIP or VIPergic nerve fibers associated with the disease of asthma. [Ollerenshaw, S. et al., New England J. Med-, 320, 1244-1248 (1989)]. In vitro and in vivo testing have shown VIP to relax tracheal smooth muscle and protect against bronchoconstrictor agents such as histamine and prostaglandin F_{2α}. [Wasserman, M. A. et al, in Vasoactive Intestinal Peptide, S. I. Said, ed., Raven Press, New York, 1982, pp 177-184; Said, S. I. et al., Ann. N.Y. Acad. Sci., 221, 103-114 (1974)]. When giving intravenously, VIP has been found to protect against bronchoconstrictor agents such as histamine, prostaglandin F_{2α}, leukotrienes, platelet activating factor as well as antigen-induced bronchoconstrictions. [Said, S. L, et al., supra, (1982)]. VIP has also been found to inhibit mucus secretion in human airway tissue in vitro. [Coles, S. J. et al. Am. Rev. Respir. Dis., 124, 531-536 (1981)].

Disorders of the airways have diverse causes but share various pathophysiologic and clinical features. Characteristic of these disorders are limitation of airflow resulting from airway obstruction, thickening of airway walls, inflammation or loss of elasticity of interstitial tissue. Co-morbidities may include hypersecretion of mucus, airway hyperreactivity, and gas exchange abnormalities which may result on cough, sputum production, wheezing and dyspnea. Common disorders of the airways include: asthma, chronic obstructive pulmonary disease (COPD), chronic bronchitis, emphysema, and pulmonary hypertension. [Mayer et al, Respiration Physiol., 128, 3-11 (2001)].

COPD is a group of chronic conditions defined by the obstruction of the lung airways. COPD includes two major breathing diseases which are chronic (obstructive) bronchitis and emphysema. Both diseases are associated with breathing difficulty and breathlessness. COPD may be accompanied by pulmonary hypertension. Long-term cigarette smoking is the predominant risk factor for COPD. The airway limitation associated with COPD is generally regarded as being irreversible.

Chronic bronchitis is a progressive inflammatory disease. Associated with this disease is an increase in mucus production in the airways and increase in the occurrence of bacterial infections. This chronic inflammatory condition induces thickening of the walls of the bronchi resulting in increased congestion and dyspnea.

Emphysema is an underlying pathology of COPD by damaging lung tissue with enlargement of the airspaces and loss of alveolar surface area. Lung damage is caused by weakening and breaking the air sacs within the lungs. Natural elasticity of the lung tissue is also lost, leading to overstretching and rupture. Smaller bronchial tubes may be damaged which can cause them to collapse and obstruct airflow, leading to shortage of breath.

COPD, in its substantial medical meaning, is always accompanied by bronchial obstruction. Thus, the most common symptoms of COPD include shortness of breath, chronic coughing, chest tightness, greater effort to breathe, increased mucus production and frequent clearing of the throat. Patients are unable to perform their usual daily activities. Independent development of chronic bronchitis and emphysema is possible, but most people with COPD have a combination of the disorders.

Breakdown of connective tissue in lung parenchyma, in particular elastin, results in the loss of elasticity found in many airway disorders. Evidence for elastin degradation has been shown in emphysema and COPD. Neutrophil elastase is considered to be a primary protease responsible for elastin destruction. [Barnes et al, Eur. Respir. J., 22, 672-688 (2003)]. Production of neutrophil elastase has been shown to be enhanced in the lungs of COPD patients. [Higashimoto et al, Respiration, 72, 629-635 (2005)].

Because of the interesting and potential clinically useful biological activities of VIP, the peptide has been the target of several reported synthetic programs with the goal of enhancing one or more of the properties of this molecule. Takeyama et al. have reported a VIP analog having a glutamic acid substituted for aspartic acid at position 8. This compound was found to be less potent than native VIP. [Chem. Pharm. Bull., 28, 2265-2269 (1980)]. Wendlberger et al. have disclosed the preparation of a VIP analog having a norleucine substituted at position 17 for methionine. [Peptide. Proc. 16th Eur. Pept. Symp., 290-295 (1980)]. The peptide was found to be equipotent to native VIP for its ability to displace radioiodinated VIP from liver membrane preparations. Watts and Wooton have reported a series of linear and cyclic VIP fragments, containing between six and twelve residues from the native sequence. [Eur. Pat. Nos. 184309 and 325044; U.S. Pat. Nos. 4,737,487 and 4,866,039]. Turner et al have reported that the fragment VIP(10-28) is an antagonist to VIP. [Peptides, 7, 849-854 (1986)]. The substituted analog [4-Cl-D-Phe⁶,Leu¹⁷]-VIP has also been reported to bind to the VIP receptor and antagonize the activity of VIP. [Pandol, S. et al., Gastrointest. Liver Physiol., 13, G553-G557 (1986)]. Gozes et al. have reported that the analog [Lys¹,Pro²,Arg³,Arg⁴,Pro⁵,Tyr⁶]-VIP is a competitive inhibitor of VIP binding to its receptor on glial cells. [Endocrinology, 125, 2945-2949 (1989)]. Robberecht, et al. have reported several VIP analogs with D-residues substituted in the N-terminus of native VIP. [Peptides, 9, 339-345 (1988)]. All of these analogs bound less tightly to the VIP receptor and showed lower activity than native VIP in c-AMP activation. Tachibana and Ito have reported several VIP analogs of the precursor molecule. [in Peptide Chem. T. Shiba and S. Sakakibara, eds., Prot. Res. Foundation, 1988, pp. 481-486, Jap. Pat. No. 1083012, U.S. Pat. No. 4,822,774]. These compounds were shown to be 1- to 3-fold more potent bronchodilators than VIP and had a 1- to 2-fold higher level of hypotensive activity. Musso et al. have also reported several VIP analogs have substitutions at positions 6-7, 9-13, 15-17, and 19-28. [Biochemistry, 27, 8174-8181 (1988); Eur. Pat. No. 8271141; U.S. Pat. No. 4,835,252]. These compounds were found to be equal to or less potent than native VIP in binding to the VIP receptor and in biological response. Bartfai et al have reported a series of multiply substituted [Leu¹⁷]-VIP analogs. [World Pat. No. 8905857].

Gourlet et al have reported an [Arg¹⁶]-VIP derivative with affinity for VIP receptors [Gourlet et al, Biochim. Biophys. Acta, 1314, 267-273 (1996)]. Onoue et al have reported a series of arginine derivatives and truncations of VIP [Onoue et al, Life Sci., 74, 1465-77 (2004) and Ohmori et al, Regul. Pept., 123, 201-7 (2004)]. A series of poly-alanine derivatives has also been reported [Igarashi et al, J. Pharm. Exper. Ther., 303, 445-60 (2002) and Igarashi et al, J. Pharm. Exper. Ther., 315, 370-81 (2005)].

Analogs of VIP having selective VPAC1 agonist activity have been reported [Pan and Roczniak, US20050203009]. Analogs of VIP and C-terminal pegylated derivatives have been reported has being of utility for the treatment of metabolic disorders including diabetes [Froland et al, WO2004006839, Clairmont et al, WO2005072385, Whelan et al, WO2005123109, Bokvist et al, WO2005113593 and WO2005113594, and Nestor, US20060079456 and WO2006042152]. Peptides having VPAC2 agonist activity have been identified, and include PACAP and VIP analogs [Gourlet, et al., Peptides 18:403-408; Xia, et al., J. Pharmacol. Exp. Ther. 281:629-633, 1997]. Cyclic analogs of VIP have been reported that have enhanced stability and activity [Bolin et al, Biopolymers, 37, 57-66 (1995) and Bolin and O'Donnell, U.S. Pat. No. 5,677,419].

In man, when administered by intravenous infusion to asthmatic patients. VIP has been shown to cause an increase in peak expiratory flow rate and protect against histamine-induced bronchodilation. [Morice, A. H. and Sever, P. S., Peptides, 7, 279-280 (1986); Morice, A. et al. The Lancet, II 1225-1227 (1983)]. The pulmonary effects observed by this intravenous infusion of VIP were, however, accompanied by cardiovascular side-effects, most notably hypotension and tachycardia and also facial flushing. When given in intravenous doses which did not cause cardiovascular effects, VIP failed to alter specific airway conductance. [Palmer, J. B. D., et al, Thorax, 41, 663-666 (1986)]. The lack of activity was explained as being due to the low dose administered and possibly due to rapid degradation of the compound. When administered by aerosol to humans, native VIP has been only marginally effective in protecting against histamine-induced bronchoconstriction. [Altieri et al., Pharmacologist, 25, 123 (1983)]. VIP was found to have no significant effect on baseline airway parameters but did have a protective effect against histamine-induced bronchoconstriction when given by inhalation to humans. [Barnes, P. J. and Dixon, C. M. S., Am. Rev. Respir. Dis. 130, 162-166 (1984)]. VIP, when given by aerosol, has been reported to display no tachycardia or hypotensive effects in conjunction with the bronchodilation. [Said, S. I et al., in Vasoactive Intestinal Peptide, S. I. Said, ed. Raven Press, New York, 1928, pp 185-191].

A derivative of VIP, RO 25-1553, has been reported to have efficacy as a bronchodilatory both preclinically and clinically in mild asthmatics [Kallstrom and Waldeck, Eur. J. Pharm., 430, 335-40 (2001) and Linden et al, Thorax, 58, 217-21 (2003)]. Native VIP has been reported to be of utility for the treatment of COPD, pulmonary hypertension and other airway disorders [WO03061680, WO0243746 and WO2005014030].

A need exists, however, for novel analogs of vasoactive intestinal peptide having selectivity for the VPAC2 receptor, while possessing equal or better potency, pharmacokinetic properties and pharmacological properties than existing VPAC agonists. Preferably, a need exists for compounds having greater duration of activity than those previously available.

### SUMMARY OF THE INVENTION

The present invention comprises a VPAC-2 receptor agonist of the formula (I): 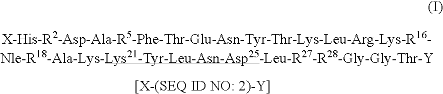
or a pharmaceutically acceptable salt thereof. Underlined residues indicate a side-chain to side-chain covalent linkage of the first and last amino acids within the segment. The present invention also encompasses pharmaceutical compositions containing such agonists, and the use of such agonists for the treatment of pulmonary diseases including COPD.

### DETAILED DESCRIPTION OF THE INVENTION

The present invention comprises a VPAC-2 receptor agonist of the formula (I): 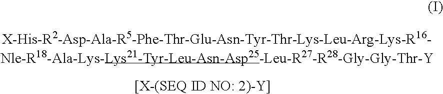
wherein:
X is a hydrogen of the N-terminal amino of Histidine which may be optionally replaced by a hydrolyzable amino protecting group, most preferably by an acetyl group,
Y is the hydroxy of the C-terminal carboxy of Threonine which may be optionally replaced by a hydrolyzable carboxy protecting group, most preferably by NH₂, underlined residues indicates a side-chain to side-chain covalent linkage of the first (Lys²¹) and last (Asp²⁵) amino acids within the segment,
R² is Ser or Ala,
R⁵ is Thr, Ser, Asp, Gln, Pro or CαMeVal,
R¹⁶ is Gln, Ala, or Arg,
R¹⁸ is Ala, Lys or Glu,
R²⁷ is Lys or Leu except that R²⁷ must be Lys when R⁵ is CαMeVal and R¹⁶ is Arg,
R²⁸ is Lys or Asn,
or a pharmaceutically acceptable salt thereof.

The compounds of the invention are active agonists of the VPAC2 receptor and have enhanced stability to human neutrophil elastase. Thus, the compounds, as selective stable analogs of native VIP having improved resistance to the effects of elastase present in the human lung, would be useful for the treatment of airway disorders, including COPD.

All peptide sequences mentioned herein are written according to the usual convention whereby the N-terminal amino acid is on the left and the C-terminal amino acid is on the right, unless noted otherwise. A short line between two amino acid residues indicates a peptide bond. A segment of amino acids with underline indicates a side-chain to side-chain covalent linkage of the first and last amino acids within the segment. Typically this is an amide bond. Where the amino acid has isomeric forms, it is the L form of the amino acid that is represented unless otherwise expressly indicated. For convenience in describing this invention, the conventional and nonconventional abbreviations for the various amino acids are used. These abbreviations are familiar to those skilled in the art, but for clarity are listed below:

Asp=D=Aspartic Acid; Ala=A=Alanine; Arg=R=Arginine; Asn=N=Asparagine; Gly=G=Glycine; Glu=E=Glutamic Acid; Gln=Q=Glutamine; His=H=Histidine; Ile=I=Isoleucine; Leu=L=Leucine; Lys=K=Lysine; Met=M=Methionine; MeVal=MeV=CαMeVal; Nle=Norleucine; Phe=F=Phenylalanine; Pro=P=Proline; Ser=S=Serine; Thr=T=Threonine; Trp=W=Tryptophan; Tyr=Y=Tyrosine; and Val=V=Valine.

With respect to the terms “hydrolyzable amino protecting group” and “hydrolyzable carboxy protecting group”, any conventional protecting groups which can be removed by hydrolysis can be utilized in accordance with this invention. Examples of such groups appear hereinafter. Preferred amino protecting groups are acyl groups of the formula 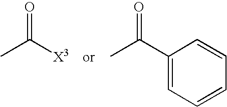
wherein X³ is lower alkyl or halo lower alkyl. Of these protecting groups, those wherein X³ is C₁₋₃ alkyl or halo C₁₋₃ alkyl are especially preferred. Preferred carboxy protecting groups are lower alkyl esters, NH₂ and lower alkyl amides, with C₁₋₃ alkyl esters, NH₂ and C₁₋₃ alkyl amides being especially preferred.

Also for convenience, and readily known to one skilled in the art, the following abbreviations or symbols are used to represent the moieties, reagents and the like used in this invention:
Fmoc 9-Fluorenylmethyloxycarbonyl;
Boc t-Butyloxycarbonyl;
Mtt 4-Methyltrityl;
2Pip 2-Phenylisopropyl ester;
Pmc 2,2,5,7,8-Pentamethylchroman-6-sulfonyl;
Nle Norleucine;
CαMeVal Cα-Methyl-L-Valine;
MeVal Cα-Methyl-L-Valine;
CH₂Cl₂ Methylene chloride;
Ac Acetyl
Ac₂O Acetic anhydride;
AcOH Acetic acid;
ACN Acetonitrile;
DMAc Dimethylacetamide;
DMF Dimethylformamide;
DIPEA N,N-Diisopropylethylamine;
TFA Trifluoroacetic acid;
HOBT N-Hydroxybenzotriazole;
DIC N,N′-Diisopropylcarbodiimide;
BOP Benzotriazol-1-yloxy-tris-(dimethylamino)phosphonium-Hexafluorophosphate;
HBTU 2-(1H-Benzotriazole-1-yl)-1,1,3,3-tetramethyluronium-Hexafluorophosphate;
NMP 1-Methyl-2-pyrrolidinone;
MALDI-TOF Matrix assisted laser desorption ionization-time of flight
FAB-MS Fast atom bombardment mass spectrometry; and
ES-MS Electro spray mass spectrometry.

As used herein, the term “alkyl” means a branched or unbranched, cyclic or acyclic, saturated or unsaturated (e.g. alkenyl or alkynyl)hydrocarbyl radical which may be substituted or unsubstituted. Where cyclic, the alkyl group is preferably C₃ to C₁₂, more preferably C₅ to C₁₀, more preferably C₅ to C₇. Where acyclic, the alkyl group is preferably C₁ to C₁₀, more preferably C₁ to C₆, more preferably methyl, ethyl, propyl (n-propyl or isopropyl), butyl (n-butyl, isobutyl or tertiary-butyl) or pentyl (including n-pentyl and isopentyl), more preferably methyl.

As used herein, the term “lower alkyl” means a branched or unbranched, cyclic or acyclic, saturated or unsaturated (e.g. alkenyl or alkynyl)hydrocarbyl radical wherein said cyclic lower alkyl group is C₅, C₆ or C₇, and wherein said acyclic lower alkyl group is C₁, C₂, C₃ or C₄, and is preferably selected from methyl, ethyl, propyl (n-propyl or isopropyl) or butyl (n-butyl, isobutyl or tertiary-butyl).

As used herein, the term “acyl” means an optionally substituted alkyl, cycloalkyl, heterocyclic, aryl or heteroaryl group bound via a carbonyl group and includes groups such as acetyl, propionyl, benzoyl, 3-pyridinylcarbonyl, 2-morpholinocarbonyl, 4-hydroxybutanoyl, 4-fluorobenzoyl, 2-naphthoyl, 2-phenylacetyl, 2-methoxyacetyl and the like.

As used herein, the term “aryl” means a substituted or unsubstituted carbocyclic aromatic group, such as phenyl or naphthyl, or a substituted or unsubstituted heteroaromatic group containing one or more, preferably one, heteroatom.

The alkyl and aryl groups may be substituted or unsubstituted. Where substituted, there will generally be 1 to 3 substituents present, preferably 1 substituent. Substituents may include: carbon-containing groups such as alkyl, aryl, arylalkyl (e.g. substituted and unsubstituted phenyl, substituted and unsubstituted benzyl); halogen atoms and halogen-containing groups such as haloalkyl (e.g. trifluoromethyl); oxygen-containing groups such as alcohols (e.g. hydroxyl, hydroxyalkyl, aryl(hydroxyl)alkyl), ethers (e.g. alkoxy, aryloxy, alkoxyalkyl, aryloxyalkyl), aldehydes (e.g. carboxaldehyde), ketones (e.g. alkylcarbonyl, alkylcarbonylalkyl, arylcarbonyl, arylalkylcarbonyl, arycarbonylalkyl), acids (e.g. carboxy, carboxyalkyl), acid derivatives such as esters (e.g. alkoxycarbonyl, alkoxycarbonylalkyl, alkylcarbonyloxy, alkylcarbonyloxyalkyl), amides (e.g. aminocarbonyl, mono- or di-alkylaminocarbonyl, aminocarbonylalkyl, mono- or di-alkylaminocarbonylalkyl, arylaminocarbonyl), carbamates (e.g. alkoxycarbonylamino, arloxycarbonylamino, aminocarbonyloxy, mono- or di-alkylaminocarbonyloxy, arylminocarbonloxy) and ureas (e.g. mono- or di-alkylaminocarbonylamino or arylaminocarbonylamino); nitrogen-containing groups such as amines (e.g. amino, mono- or di-alkylamino, aminoalkyl, mono- or di-alkylaminoalkyl), azides, nitriles (e.g. cyano, cyanoalkyl), nitro; sulfur-containing groups such asthiols, thioethers, sulfoxides and sulfones (e.g. alkylthio, alkylsulfinyl, alkylsulfonyl, alkylthioalkyl, alkylsulfinylalkyl, alkylsulfonylalkyl, arylthio, arysulfinyl, arysulfonyl, arythioalkyl, arylsulfinylalkyl, arylsulfonylalkyl); and heterocyclic groups containing one or more, preferably one, heteroatom.

As used herein, the term “halogen” means a fluorine, chlorine, bromine or iodine radical, preferably a fluorine, chlorine or bromine radical, and more preferably a fluorine or chlorine radical.

“Pharmaceutically acceptable salt” refers to conventional acid-addition salts or base-addition salts that retain the biological effectiveness and properties of the compounds of formula I and are formed from suitable non-toxic organic or inorganic acids or organic or inorganic bases. Sample acid-addition salts include those derived from inorganic acids such as hydrochloric acid, hydrobromic acid, hydroiodic acid, sulfuric acid, sulfamic acid, phosphoric acid and nitric acid, and those derived from organic acids such as p-toluenesulfonic acid, salicylic acid, methanesulfonic acid, oxalic acid, succinic acid, citric acid, malic acid, lactic acid, fumaric acid, and the like. Sample base-addition salts include those derived from ammonium, potassium, sodium and, quaternary ammonium hydroxides, such as for example, tetramethylammonium hydroxide. The chemical modification of a pharmaceutical compound (i.e. drug) into a salt is a well known technique which is used in attempting to improve properties involving physical or chemical stability, e.g., hygroscopicity, flowability or solubility of compounds. See, e.g., H. Ansel et. al., Pharmaceutical Dosage Forms and Drug Delivery Systems (6th Ed. 1995) at pp. 196 and 1456-1457.

“Pharmaceutically acceptable ester” refers to a conventionally esterified compound of formula I having a carboxyl group, which esters retain the biological effectiveness and properties of the compounds of formula I and are cleaved in vivo (in the organism) to the corresponding active carboxylic acid. Examples of ester groups which are cleaved (in this case hydrolyzed) in vivo to the corresponding carboxylic acids are those in which the cleaved hydrogen is replaced with -lower alkyl which is optionally substituted, e.g., with heterocycle, cycloalkyl, etc. Examples of substituted lower alkyl esters are those in which -lower alkyl is substituted with pyrrolidine, piperidine, morpholine, N-methylpiperazine, etc. The group which is cleaved in vivo may be, for example, ethyl, morpholino ethyl, and diethylamino ethyl. In connection with the present invention, —CONH₂ is also considered an ester, as the —NH₂ is cleaved in vivo and replaced with a hydroxy group, to form the corresponding carboxylic acid.

Further information concerning examples of and the use of esters for the delivery of pharmaceutical compounds is available in Design of Prodrugs, Bundgaard H. ed. (Elsevier, 1985). See also, H. Ansel et. al., Pharmaceutical Dosage Forms and Drug Delivery Systems (6th Ed. 1995) at pp. 108-109; Krogsgaard-Larsen, et. al., Textbook of Drug Design and Development (2d Ed. 1996) at pp. 152-191

The present representative compounds may be readily synthesized by any known conventional procedure for the formation of a peptide linkage between amino acids. Such conventional procedures include, for example, any solution phase procedure permitting a condensation between the free alpha amino group of an amino acid or residue thereof having its carboxyl group and other reactive groups protected and the free primary carboxyl group of another amino acid or residue thereof having its amino group or other reactive groups protected.

Such conventional procedures for synthesizing the novel compounds of the present invention include for example any solid phase peptide synthesis method. In such a method the synthesis of the novel compounds can be carried out by sequentially incorporating the desired amino acid residues one at a time into the growing peptide chain according to the general principles of solid phase methods. Such methods are disclosed in, for example, Merrifield, R. B., J. Amer. Chem. Soc. 85, 2149-2154 (1963); Barany et al., The Peptides, Analysis, Synthesis and Biology, Vol. 2, Gross, E. and Meienhofer, J., Eds. Academic Press 1-284 (1980), which are incorporated herein by reference. Peptide synthesis may be performed manually or with automated instrumentation. Microwave-assisted synthesis may also be utilized.

Common to chemical syntheses of peptides is the protection of reactive side chain groups of the various amino acid moieties with suitable protecting groups, which will prevent a chemical reaction from occurring at that site until the protecting group is ultimately removed. Usually also common is the protection of the alpha amino group on an amino acid or fragment while that entity reacts at the carboxyl group, followed by the selective removal of the alpha amino protecting group at allow a subsequent reaction to take place at that site. While specific protecting groups have been disclosed in regard to the solid phase synthesis method, it should be noted that each amino acid can be protected by a protective group conventionally used for the respective amino acid in solution phase synthesis.

Alpha amino groups may be protected by a suitable protecting group selected from aromatic urethane-type protecting groups, such as allyloxycarbonyl, benzyloxycarbonyl (Z) and substituted benzyloxycarbonyl, such as p-chlorobenzyloxycarbonyl, p-nitrobenzyloxycarbonyl, p-bromobenzyloxycarbonyl, p-biphenyl-isopropyloxycarbonyl, 9-fluorenylmethyloxycarbonyl (Fmoc) and p-methoxybenzyloxycarbonyl (Moz); aliphatic urethane-type protecting groups, such as t-butyloxycarbonyl (Boc), diisopropylmethyloxycarbonyl, isopropyloxycarbonyl, and allyloxycarbonyl. Herein, Fmoc is most preferred for alpha amino protection.

Guanidino groups may be protected by a suitable protecting group selected from nitro, p-toluenesulfonyl (Tos), (Z,) pentamethylchromanesulfonyl (Pmc), 4-methoxy-2,3,6,-trimethylbenzenesulfonyl (Mtr). Pmc and Mtr are most preferred for arginine (Arg).

The ε-amino groups may be protected by a suitable protecting group selected from 2-chloro-benzyloxycarbonyl (2-Cl-Z),2-bromo-benzyloxycarbonyl (2-Br-Z)- and t-butyloxycarbonyl (Boc). Boc is the most preferred for (Lys).

Hydroxyl groups (OH) may be protected by a suitable protecting group selected from benzyl (Bzl), 2,6 dichlorobenzyl (2,6-diCl-Bzl), and tert-butyl (t-Bu). tBu is most preferred for (Tyr), (Ser) and (Thr).

The β- and γ-amide groups may be protected by a suitable protecting group selected from 4-methyltrityl (Mtt), 2,4,6-trimethoxybenzyl (Tmob), 4,4-dimethoxydityl/bis-(4-methoxyphenyl)-methyl (Dod) and trityl (Trt). Trt is the most preferred for (Asn) and (Gln).

The indole group may be protected by a suitable protecting group selected from formyl (For), mesityl-2-sulfonyl (Mts) and t-butyloxycarbonyl (Boc). Boc is the most preferred for (Trp).

The β- and γ-carboxyl groups may be protected by a suitable protecting group selected from t-butyl (tBu), and 2-phenylisopropyl (2Pip). tBu is the most preferred for (Glu) and 2Pip is most preferred for (Asp).

The imidazole group may be protected by a suitable protecting group selected from benzyl (Bzl), t-butyloxycarbonyl (Boc), and trityl (Trt). Trt is the most preferred for (His).

All solvents, isopropanol (iPrOH), methylene chloride (CH₂Cl₂), dimethylformamide (DMF) and N-methylpyrrolinone (NMP) were purchased from Fisher, J T Baker or Burdick & Jackson and were used without additional distillation. Trifluoroacetic acid was purchased from Halocarbon, Aldrich or Fluka and used without further purification.

Diisopropylcarbodiimide (DIC) and diisopropylethylamine (DIPEA) was purchased from Fluka or Aldrich and used without further purification. Hydroxybenzotriazole (HOBT) dimethylsulfide (DMS) and 1,2-ethanedithiol (EDT) were purchased from Aldrich, Sigma Chemical Co. or Anaspec and used without further purification. Protected amino acids were generally of the L configuration and were obtained commercially from Bachem, Advanced ChemTech, CEM or Neosystem. Purity of these reagents was confirmed by thin layer chromatography, NMR and melting point prior to use. Benzhydrylamine resin (BHA) was a copolymer of styrene-1% divinylbenzene (100-200 or 200-400 mesh) obtained from Bachem, Anaspec or Advanced Chemtech. Total nitrogen content of these resins were generally between 0.3-1.2 meq/g.

High performance liquid chromatography (HPLC) was conducted on a LDC apparatus consisting of Constametric I and III pumps, a Gradient Master solvent programmer and mixer, and a Spectromonitor III variable wavelength UV detector. Analytical HPLC was performed in reversed phase mode using Pursuit C₁₈ columns (4.5×50 mm). Preparative HPLC separations were run on Pursuit columns (50×250 mm).

In a preferred embodiment, peptides were prepared using solid phase synthesis by the method generally described by Merrifield, (J. Amer. Chem. Soc., 85, 2149 (1963)), although other equivalent chemical synthesis known in the art could be used as previously mentioned. Solid phase synthesis is commenced from the C-terminal end of the peptide by coupling a protected alpha-amino acid to a suitable resin. Such a starting material can be prepared by attaching an alpha-amino-protected amino acid by an ester linkage to a p-benzyloxybenzyl alcohol (Wang) resin, or by an amide bond between an Fmoc-Linker, such as p-((R, S)-α-(1-(9H-fluoren-9-yl)-methoxyformamido)-2,4-dimethyloxybenzyl)-phenoxyacetic acid (Rink linker) to a benzhydrylamine (BHA) resin. Preparation of the hydroxymethyl resin is well known in the art. Fmoc-Linker-BHA resin supports are commercially available and generally used when the desired peptide being synthesized has an unsubstituted amide at the C-terminus.

Typically, the amino acids or mimetic are coupled onto the Fmoc-Linker-BHA resin using the Fmoc protected form of amino acid or mimetic, with 1-5 equivalents of amino acid and a suitable coupling reagent. After couplings, the resin may be washed and dried under vacuum. Loading of the amino acid onto the resin may be determined by amino acid analysis of an aliquot of Fmoc-amino acid resin or by determination of Fmoc groups by UV analysis. Any unreacted amino groups may be capped by treating the resin with acetic anhydride and diispropylethylamine in methylene chloride or DMF.

The resins are carried through several repetitive cycles to add amino acids sequentially. The alpha amino Fmoc protecting groups are removed under basic conditions. Piperidine, piperazine or morpholine (20-40% v/v) in DMF may be used for this purpose. Preferably 40% piperidine in DMF is typically utilized

Following the removal of the alpha amino protecting group, the subsequent protected amino acids are coupled stepwise in the desired order to obtain an intermediate, protected peptide-resin. The activating reagents used for coupling of the amino acids in the solid phase synthesis of the peptides are well known in the art. For example, appropriate reagents for such syntheses are benzotriazol-1-yloxy-tri-(dimethylamino)phosphonium hexafluorophosphate (BOP), Bromo-tris-pyrrolidino-phosphonium hexafluorophosphate (PyBroP) 2-(1H-Benzotriazole-1-yl)-1,1,3,3-tetramethyluronium hexafluorophosphate (HBTU), and diisopropylcarbodiimide (DIC). Preferred here are HBTU and DIC. Other activating agents are described by Barany and Merrifield (in The Peptides, Vol. 2, J. Meienhofer, ed., Academic Press, 1979, pp 1-284) may be utilized. Various reagents such as 1 hydroxybenzotriazole (HOBT), N-hydroxysuccinimide (HOSu) and 3,4-dihydro-3-hydroxy-4-oxo-1,2,3-benzotriazine (HOOBT) may be added to the coupling mixtures in order to optimize the synthetic cycles. Preferred here is HOBT.

The protocol for a typical synthetic cycle is as follows:

Protocol 1

| Step | Reagent | Time |
|---|---|---|
| 1 | DMF | 2 × 30 sec |
| 2 | 20% piperidine/DMF | 1 min |
| 3 | 20% piperidine/DMF | 15 min |
| 4 | DMF | 2 × 30 sec |
| 5 | iPrOH | 2 × 30 sec |
| 6 | DMF | 3 × 30 sec |
| 7 | Coupling | 60 min-18 hours |
| 8 | DMF | 2 × 30 sec |
| 9 | iPrOH | 1 × 30 sec |
| 10 | DMF | 1 × 30 sec |
| 11 | CH₂Cl₂ | 2 × 30 sec. |

Solvents for all washings and couplings were measured to volumes of 10-20 ml/g resins. Coupling reactions throughout the synthesis were monitored by the Kaiser ninhydrin test to determine extent of completion (Kaiser et al, Anal. Biochem., 34, 595-598 (1970)). Any incomplete coupling reactions were either recoupled with freshly prepared activated amino acid or capped by treating the peptide resin with acetic anhydride as described above. The fully assembled peptide-resins were dried in vacuum for several hours.

Peptide synthesis may be performed using an Applied Biosystem 433A synthesizer (Foster City, Calif.). The FastMoc 0.25 mmole cycles were used with either the resin sampling or non resin sampling, 41 mL reaction vessel. The Fmoc-amino acid resin was dissolved with 2.1 g NMP, 2 g of 0.45M HOBT/HBTU in DMF and 2M DIEA, then transferred to the reaction vessel. The basic FastMoc coupling cycle was represented by the module “BADEIFD,” wherein each letter represents a module. For example: B represents the module for Fmoc deprotection using 20% piperidine/NMP and related washes and readings for 30 min (either UV monitoring or conductivity); A represents the module for activation of amino acid in cartridges with 0.45 M HBTU/HOBt and 2.0 M DIEA and mixing with N2 bubbling; D represents the module for NMP washing of resin in the reaction vessel; E represents the module for transfer of the activated amino acid to the reaction vessel for coupling; I represents the module for a 10 minute waiting period with vortexing on and off of the reaction vessel; and F represents the module for cleaning cartridge, coupling for approximately 10 minutes and draining the reaction vessel. Couplings were typically extended by addition of module “I” once or multiple times. For example, double couplings were run by performing the procedure “BADEIIADEIFD.” Other modules were available such as c for methylene chloride washes and “C” for capping with acetic anhydride. Individual modules were also modifiable by, for example, changing the timing of various functions, such as transfer time, in order to alter the amount of solvent or reagents transferred. The cycles above were typically used for coupling one amino acid. For synthesizing tetra peptides, however, the cycles were repeated and strung together. For example, BADEIIADEIFD was used to couple the first amino acid, followed by BADEIIADEIFD to couple the second amino acid, followed by BADEIIADEIFD to couple the third amino acid, followed by BADEIIADEIFD to couple the fourth amino acid, followed by BIDDcc for final deprotection and washing.

Peptide synthesis may be performed using a Microwave Peptide Synthesizer, Liberty (CEM Corporation, Matthews, N.C.). The synthesizer was programmed for double coupling and capping by modification of preloaded 0.25 mmol cycle. The microwave editor was used to program microwave power methods for use during the Fmoc deprotection, amino acid coupling and capping with acetic anhydride. This type of microwave control allows for methods to be created that control a reaction at a set temperature for a set amount of time. The Liberty automatically regulates the amount of power delivered to the reaction to keep the temperature at the set point. The default cycles for amino acid addition and final deprotection were selected in cycle editor and were automatically loaded while creating a peptide.

The synthesis was carried out on a 0.25 mmol scale using Fmoc-Linker-BHA resin (450 mg, 0.25 mmol). Resin was added to the 30 mL reaction vessel with 10 mL of DMF. Fmoc deprotection was performed with a 20% piperidine in DMF solution. For each amino acid coupling, Fmoc protected amino acid was dissolved in DMF to make a 0.2M solution and was added to the reaction vessel. All coupling reactions were performed with 0.5M HOBT/HBTU and 2M DIEA/NMP. Any incomplete coupling reactions were either recoupled with freshly prepared activated amino acid or capped by treating the peptide resin with 25% acetic anhydride in DMF. Each deprotection, coupling and capping reaction was done using Microwave at 70° C. for 300 seconds at 50 watts power and nitrogen bubbling.

For each Amino acid coupling following 0.25 mmol coupling cycle was used:

Protocol 2

Transfer resin to vessel

Add Piperidine Deprotection (10 mL)

Microwave method for deprotection (50 watts; 70° C.; 300 seconds)

Wash resin with DMF (10 mL)

Add Amino acid (5 mL)

Add Activator (HOBT/HBTU) (2 mL)

Add Activator base (DIEA) (1 mL)

Microwave method for Coupling (50 watts; 70° C.; 300 seconds)

Wash resin with DMF (10 mL)

Add Amino acid (5 mL)

Add Activator (HOBT/HBTU) (2 mL)

Add Activator base (DIEA) (1 mL)

Microwave method for Coupling (50 watts; 70° C.; 300 seconds)

Wash resin with DMF (10 mL)

Add capping (Acetic Anhydride 10 mL)

Microwave Method (capping) (50 watts; 70° C.; 300 seconds)

Wash resin with DMF (10 mL)

For synthesis of compounds presented here, a preferred synthetic procedure is shown in Scheme 1.

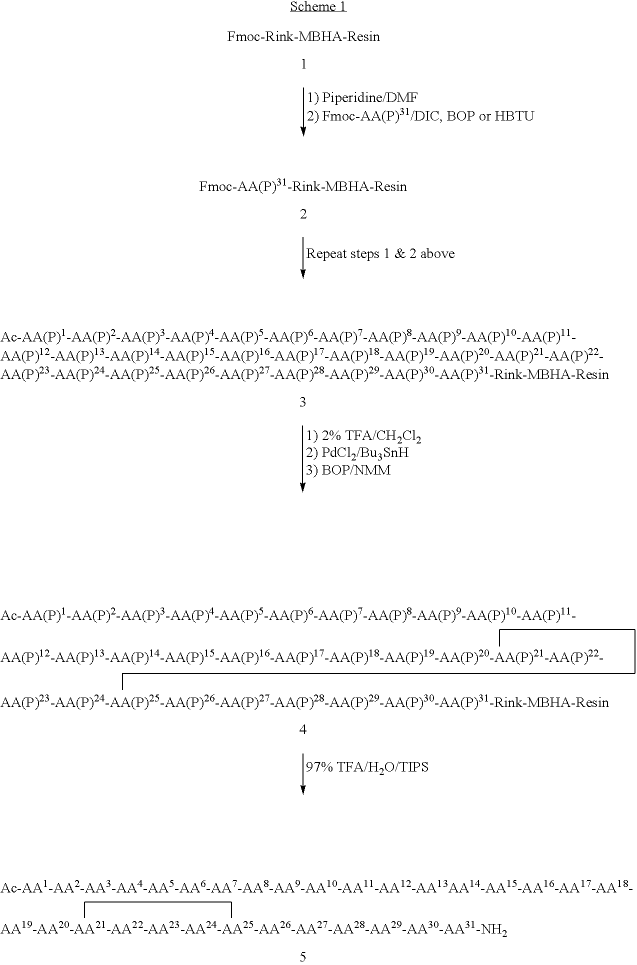

Treatment of Fmoc-Rink-MBHA resin, 1, with piperidine/DMF followed by coupling with Fmoc-AA(P)³¹ with a reagent such as DIC, BOP or HBTU, where AA31 represents the 31^{st} amino acid residue and P represents an appropriate protecting group, yields Fmoc-AA(P)³¹-Rink-Resin, 2. Repetition of steps 1 & 2 for 30 cycles by adding the appropriate protected amino acid at each cycle, yields peptide resin 3. The side chain protecting groups on AA²⁵ and AA²¹ are removed by treatment with 2% TFA in CH₂Cl₂ and PdCl₂/nBu₃SnH, respectively. The side chain amine and carboxyl of AA²¹ and AA²⁵ are cyclized by treatment with BOP and NMM in DMF to yield 4.

For each compound, the blocking groups are removed and the peptide cleaved from the resin in the same step. For example, the peptide-resins may be treated with 100 μL ethanedithiol, 100 μl dimethylsulfide, 300 μL anisole, and 9.5 mL trifluoroacetic acid, per gram of resin, at room temperature for 180 min. Or alternately, the peptide-resins may be treated with 1.0 mL triisopropyl silane and 9.5 mL trifluoroacetic acid, per gram of resin, at room temperature for 180 min. The resin is filtered off and the filtrates are precipitated in chilled ethyl ether. The precipitates are centrifuged and the ether layer is decanted. The residue was washed with two or three volumes of Et₂O and re-centrifuged. The crude product 5 is dried under vacuum.

Purifications of the crude peptides were performed on Shimadzu LC-8A system by high performance liquid chromatography (HPLC) on a reverse phase Pursuit C-18 Column (50×250 mm. 300 A°, 10 um). The peptides were dissolved in a minimum amount of water and Acetonitrile and were injected in a column. Gradient elution was generally started at 2% B buffer, 2%-70% B over 70 minutes, (buffer A: 0.1% TFA/H₂O, buffer B: 0.1% TFA/CH₃CN) at a flow rate of 50 ml/min. UV detection was made at 220/280 nm. The fractions containing the products were separated and their purity was judged on Shimadzu LC-10AT analytical system using reverse phase Pursuit C18 column (4.6×50 mm) at a flow rate of 2.5 ml/min., gradient (2-70%) over 10 min. [buffer A: 0.1% TFA/H₂O, buffer B: 0.1% TFA/CH₃CN)]. Fractions judged to be of sufficient purity were pooled and lyophilized.

Purity of the final products was checked by analytical HPLC on a reversed phase column as stated above. All final products were also subjected to fast atom bombardment mass spectrometry (FAB-MS) or electrospray mass spectrometry (ES-MS). All products yielded the expected parent M+H ions within acceptable limits.

Analogs of VIP described in the invention are agonists of the VPAC2 receptor as demonstrated in Example X. According to the elastase stability experiments in Example X, such compounds have enhanced stability to human neutrophil elastase. Therefore, administration of these VPAC2 receptor agonists would be of utility for the treatment of airway disorders such as COPD.

The compounds of the present invention can be provided in the form of pharmaceutically acceptable salts. Examples of preferred salts are those formed with pharmaceutically acceptable organic acids, e.g., acetic, lactic, maleic, citric, malic, ascorbic, succinic, benzoic, salicylic, methanesulfonic, toluenesulfonic, trifluoroacetic, or pamoic acid, as well as polymeric acids such as tannic acid or carboxymethyl cellulose, and salts with inorganic acids, such as hydrohalic acids (e.g., hydrochloric acid), sulfuric acid, or phosphoric acid and the like. Any procedure for obtaining a pharmaceutically acceptable salt known to a skilled artisan can be used.

In the practice of the method of the present invention, an effective amount of any one of the peptides of this invention or a combination of any of the peptides of this invention or a pharmaceutically acceptable salt thereof, is administered via any of the usual and acceptable methods known in the art, either singly or in combination. The compounds or compositions can thus be administered orally (e.g., buccal cavity), sublingually, parenterally (e.g., intramuscularly, intravenously, or subcutaneously), rectally (e.g., by suppositories or washings), transdermally (e.g., skin electroporation) or by inhalation (e.g., by aerosol), and in the form of solid, liquid or gaseous dosages, including tablets and suspensions. The administration can be conducted in a single unit dosage form with continuous therapy or in a single dose therapy ad libitum. The therapeutic composition can also be in the form of an oil emulsion or dispersion in conjunction with a lipophilic salt such as pamoic acid, or in the form of a biodegradable sustained-release composition for subcutaneous or intramuscular administration.

Thus, the method of the present invention is practiced when relief of symptoms is specifically required or perhaps imminent. Alternatively, the method of the present invention is effectively practiced as continuous or prophylactic treatment.

Useful pharmaceutical carriers for the preparation of the compositions hereof, can be solids, liquids or gases; thus, the compositions can take the form of tablets, pills, capsules, suppositories, powders, enterically coated or other protected formulations (e.g. binding on ion-exchange resins or packaging in lipid-protein vesicles), sustained release formulations, solutions, suspensions, elixirs, aerosols, and the like. The carrier can be selected from the various oils including those of petroleum, animal, vegetable or synthetic origin, e.g., peanut oil, soybean oil, mineral oil, sesame oil, and the like. Water, saline, aqueous dextrose, and glycols are preferred liquid carriers, particularly (when isotonic with the blood) for injectable solutions. For example, formulations for intravenous administration comprise sterile aqueous solutions of the active ingredient(s) which are prepared by dissolving solid active ingredient(s) in water to produce an aqueous solution, and rendering the solution sterile. Suitable pharmaceutical excipients include starch, cellulose, talc, glucose, lactose, gelatin, malt, rice, flour, chalk, silica, magnesium stearate, sodium stearate, glycerol monostearate, sodium chloride, dried skim milk, glycerol, propylene glycol, water, ethanol, and the like. The compositions may be subjected to conventional pharmaceutical additives such as preservatives, stabilizing agents, wetting or emulsifying agents, salts for adjusting osmotic pressure, buffers and the like. Suitable pharmaceutical carriers and their formulation are described in Remington's Pharmaceutical Sciences by E. W. Martin. Such compositions will, in any event, contain an effective amount of the active compound together with a suitable carrier so as to prepare the proper dosage form for proper administration to the recipient.

The dose of a compound of the present invention depends on a number of factors, such as, for example, the manner of administration, the age and the body weight of the subject, and the condition of the subject to be treated, and ultimately will be decided by the attending physician or veterinarian. Such an amount of the active compound as determined by the attending physician or veterinarian is referred to herein, and in the claims, as an “effective amount”. For example, the dose for inhalation administration is typically in the range of about 0.5 to about 100 μg/kg body weight. Preferably, the compound of the present invention is administered at a dose rate of from about 1 μg/kg to about 50 μg/kg/day.

Representative delivery regimens include oral, parenteral (including subcutaneous, intramuscular and intravenous), rectal, buccal (including sublingual), transdermal, pulmonary and intranasal. The preferred route of administration is pulmonary administration by oral inhalation. Methods of pulmonary administration may include aerosolization of an aqueous solution of the cyclic peptides of the present invention or the inspiration of micronized dry powder formulations. Aerosolized compositions may include the compound packaged in reverse micelles or liposomes. The preparation of micronized powders of suitably controlled particle size to effectively provide for alveolar delivery is well known. Inhalers for the delivery of specified doses of such formulations directly into the lungs (Metered Dose Inhalers or “MDIs”) are well known in the art.

The invention will now be further described in the following Examples, which are intended as an illustration only and do not limit the scope of the invention.

### EXAMPLES

### Example 1

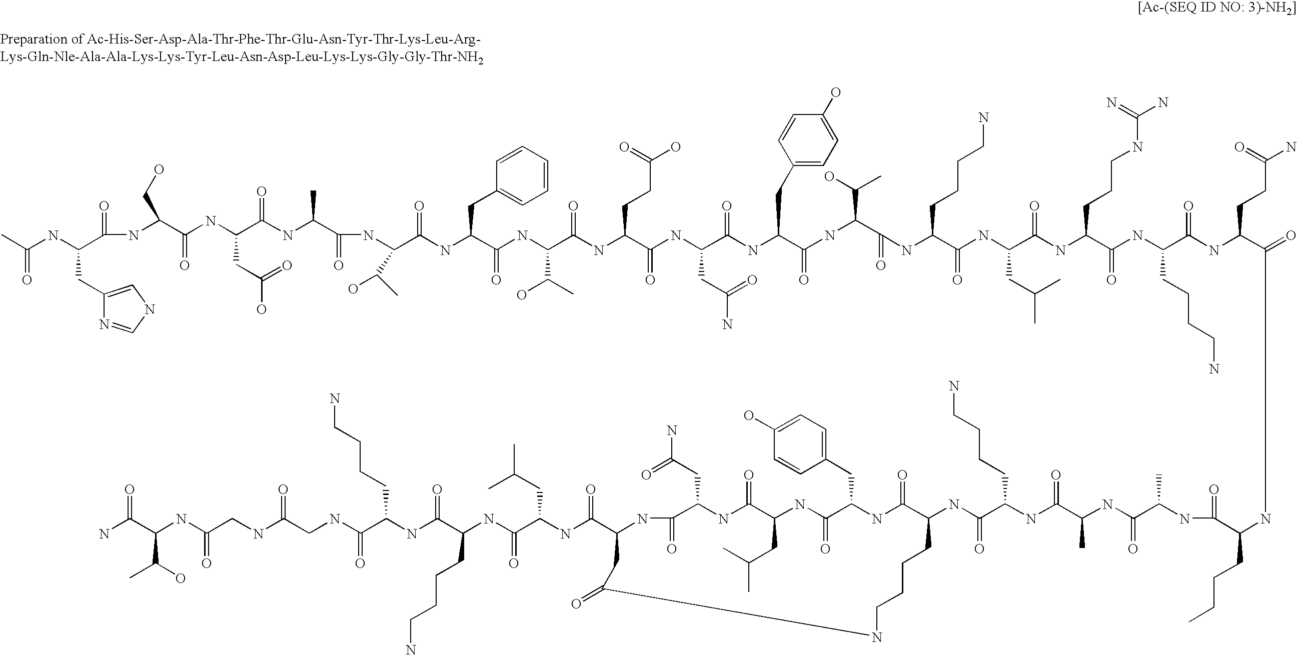

The above peptide was synthesized using Fmoc chemistry on an Applied Biosystem 433A or a microwave Peptide synthesizer. The synthesizer was programmed for double coupling using the modules described in Protocol 1 or 2 above. The synthesis was carried out on a 0.25 mmol scale using the Fmoc-Rink Linker-BHA resin (450 mg, 0.25 mmol). At the end of the synthesis, the resin was transferred to a reaction vessel on a shaker. The peptide resin in DMF was filtered and washed with CH₂Cl₂. The resin was treated five times with 2% TFA in CH₂Cl₂ for 3 min each. The resin was immediately treated twice with 5% DIPEA/CH₂Cl₂ and washed with CH₂Cl₂ and DMF. The peptide resin was suspended in DMF in a shaker vessel securely fitted with a rubber septum. To this was added 60 mg PdCl₂(Ph₃P)₂, 150 uL morpholine and 300 uL AcOH. The vessel was purged well with Ar. nBu₃SnH was then added via syringe. The black solution was shaken for 30-45 minutes, washed with DMF and repeated. Following the second Pd treatment, the resin was washed with DMF, 2×iPrOH, DMF, 5% DIPEA/DMF and DMF. In DMF, the peptide resin was cyclized by treatment with BOP and NMM overnight. The resin was washed with DMF and CH₂Cl₂ and then dried under vacuum.

The peptide was cleaved from the resin using 13.5 mL 97% TFA/3% H₂O and 1.5 mL triisopropylsilane for 180 minutes at room temperature. The deprotection solution was added to 100 mL cold Et₂O, and washed with 1 mL TFA and 30 mL cold Et₂O to precipitate the peptide. The peptide was centrifuged in two 50 mL polypropylene tubes. The precipitates from the individual tubes were combined in a single tube and washed 3 times with cold Et₂O and dried in a desiccator under house vacuum.

The crude material was purified by preparative HPLC on a Pursuit C18-Column (250×50 mm, 10 μm particle size) and eluted with a linear gradient of 2-70% B (buffer A: 0.1% TFA/H₂O; buffer B: 0.1% TFA/CH₃CN) in 90 min., flow rate 60 mL/min, and detection 220/280 nm. The fractions were collected and were checked by analytical HPLC. Fractions containing pure product were combined and lyophilized to yield 106 mg (9.7%) of a white amorphous powder. (ES)+-LCMS m/e calculated (“calcd”) for C₁₅₉H₂₅₆N₄₆O₄₇ 3565.05 found 3563.7.

### Example 2

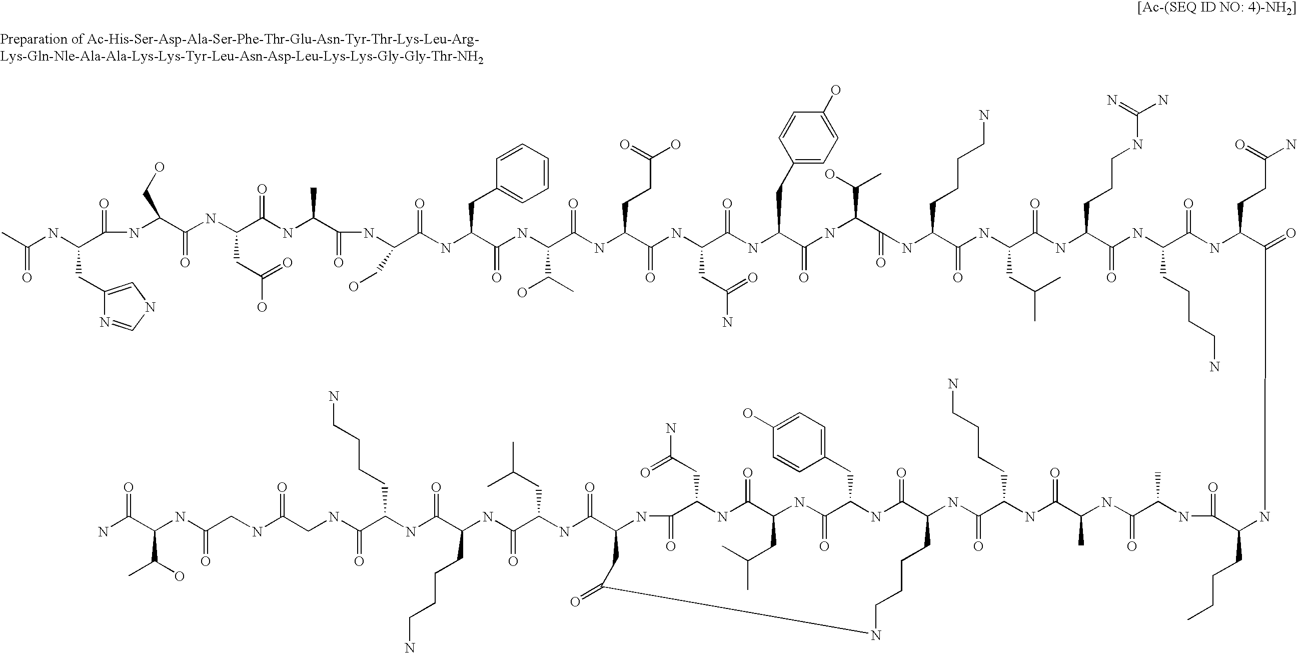

Fmoc-Rink-Linker-BHA resin (450 mg, 0.25 mmol) was subjected to solid phase synthesis and purification by following the procedure in Example 1 to yield 28 mg (2.5%) of white amorphous powder. (ES)+-LCMS m/e calcd for C₁₅₈H₂₅₄N₄₆O₄₇ 3551.02 found 3548.7.

### Example 3

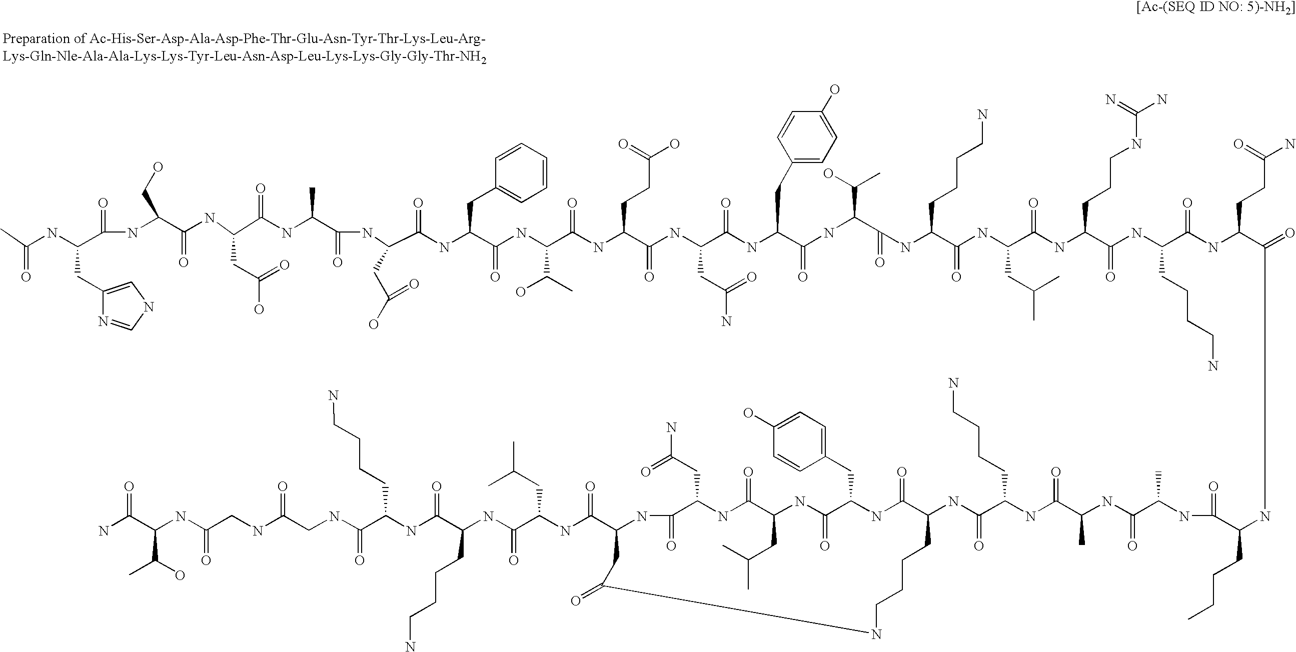

Fmoc-Rink-Linker-BHA resin (450 mg, 0.25 mmol) was subjected to solid phase synthesis and purification by following the procedure in Example 1 to yield 9.2 mg (1%) of white amorphous powder. (ES)+-LCMS m/e calcd for C₁₅₉H₂₅₄N₄₆O₄₈ 3579.03 found 3577.8.

### Example 4

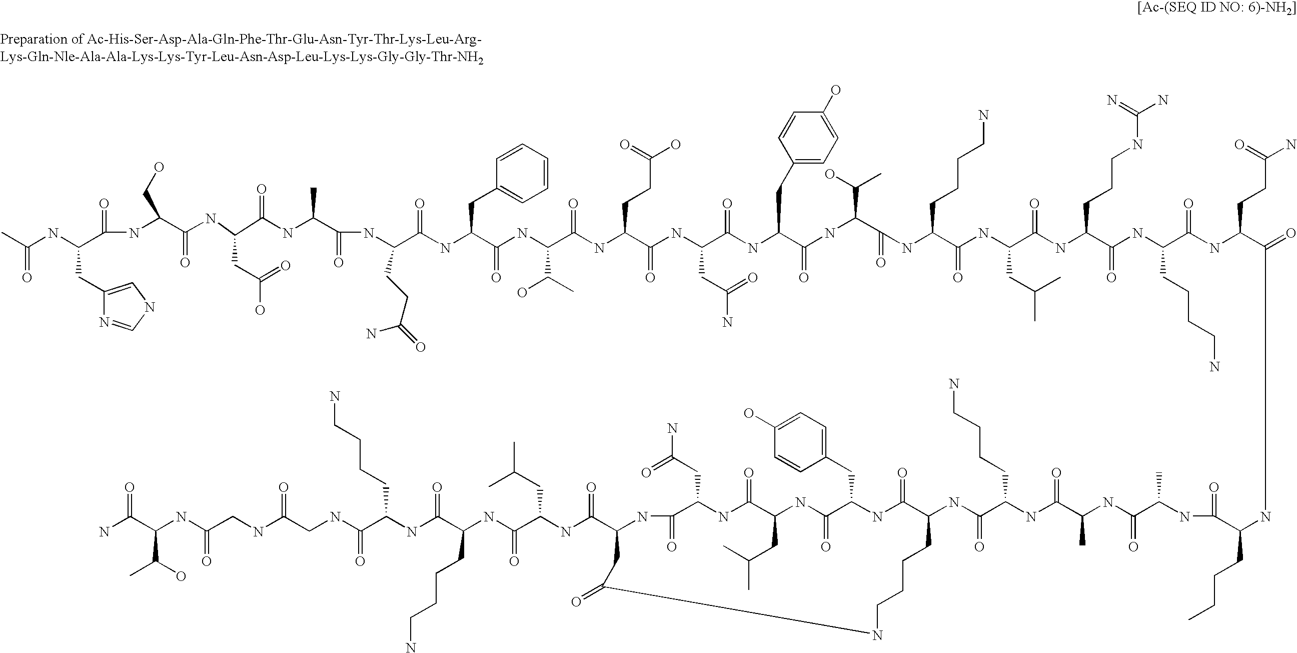

Fmoc-Rink-Linker-BHA resin (450 mg, 0.25 mmol) was subjected to solid phase synthesis and purification by following the procedure in Example 1 to yield 9.8 mg (1%) of white amorphous powder. (ES)+-LCMS m/e calcd for C₁₆₀H₂₅₇N₄₇O₄₇ 3592.07 found 3589.5.

### Example 5

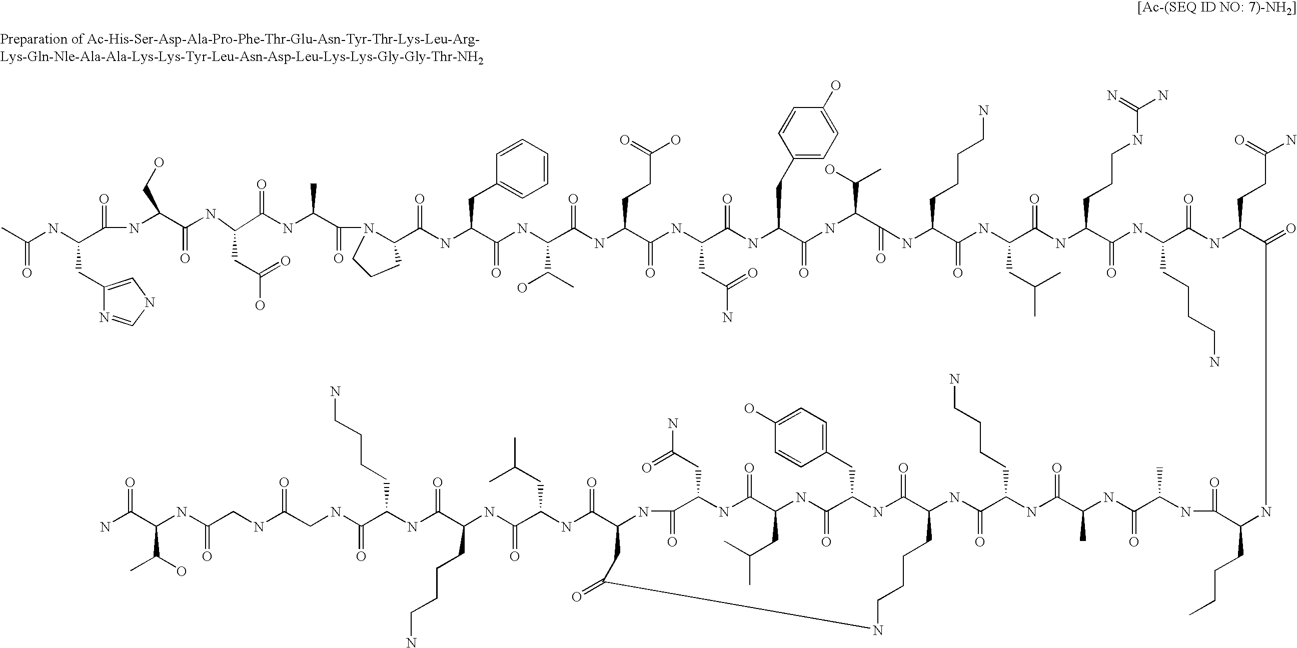

Fmoc-Rink-Linker-BHA resin (450 mg, 0.25 mmol) was subjected to solid phase synthesis and purification by following the procedure in Example 1 to yield 15.2 mg (1.4%) of white amorphous powder. (ES)+-LCMS m/e calcd for C₁₆₀H₂₅₆N₄₆O₄₆ 3561.06 found 3560.0.

### Example 6

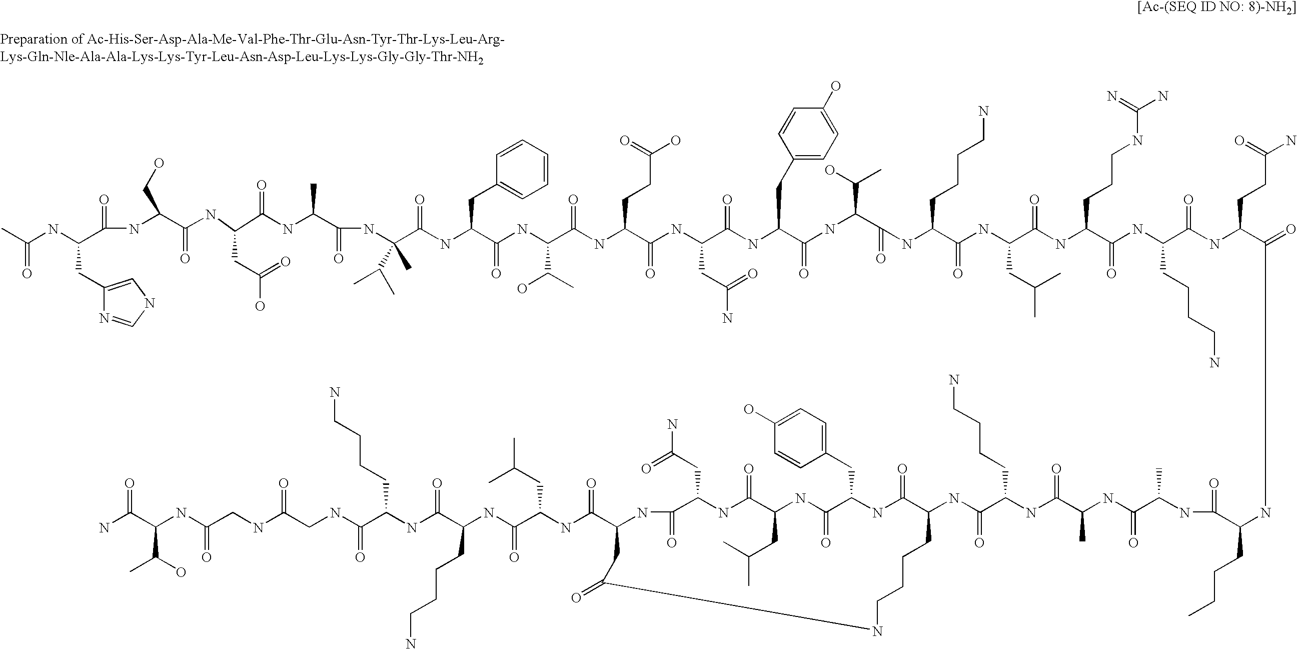

Fmoc-Rink-Linker-BHA resin (450 mg, 0.25 mmol) was subjected to solid phase synthesis and purification by following the procedure in Example 1 to yield 40 mg (3.6%) of white amorphous powder. (ES)+-LCMS m/e calcd for C₁₆₁H₂₆₀N₄₆O₄₆ 3577.10 found 3576.8.

### Example 7

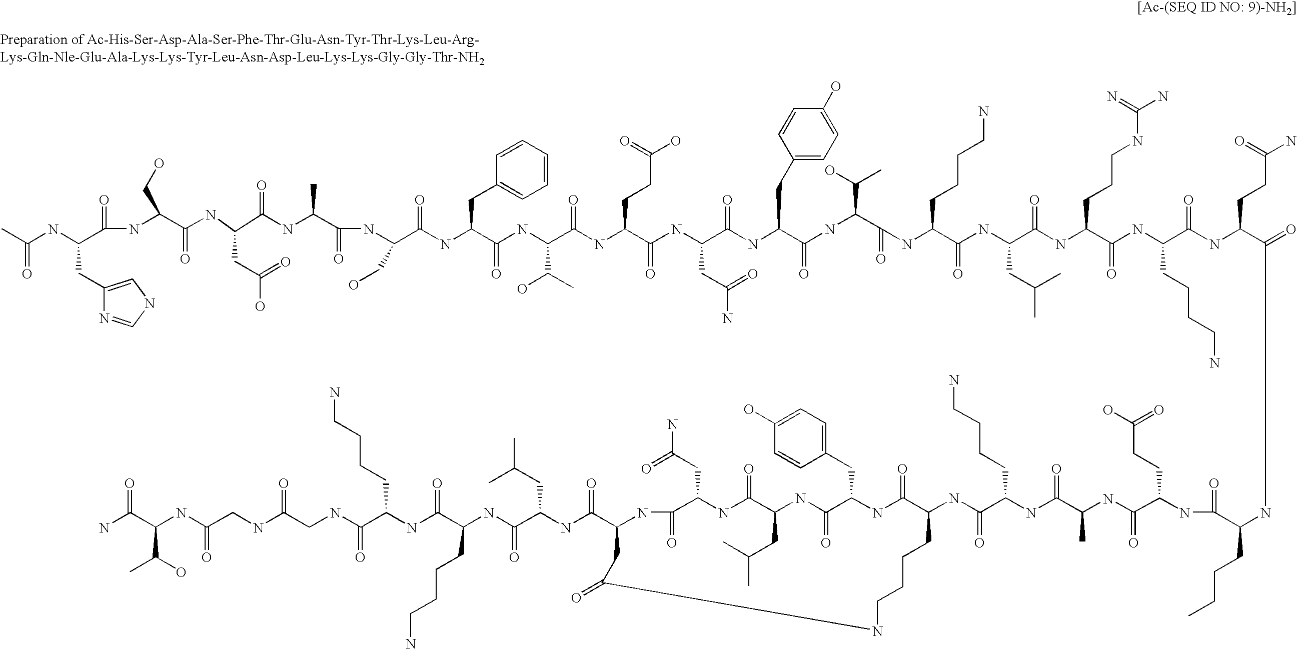

Fmoc-Rink-Linker-BHA resin (450 mg, 0.25 mmol) was subjected to solid phase synthesis and purification by following the procedure in Example 1 to yield 126 mg (11.4%) of white amorphous powder. (ES)+-LCMS m/e calcd for C₁₆₀H₂₅₆N₄₆O₄₉ 3609.06 found 3609.2.

### Example 8

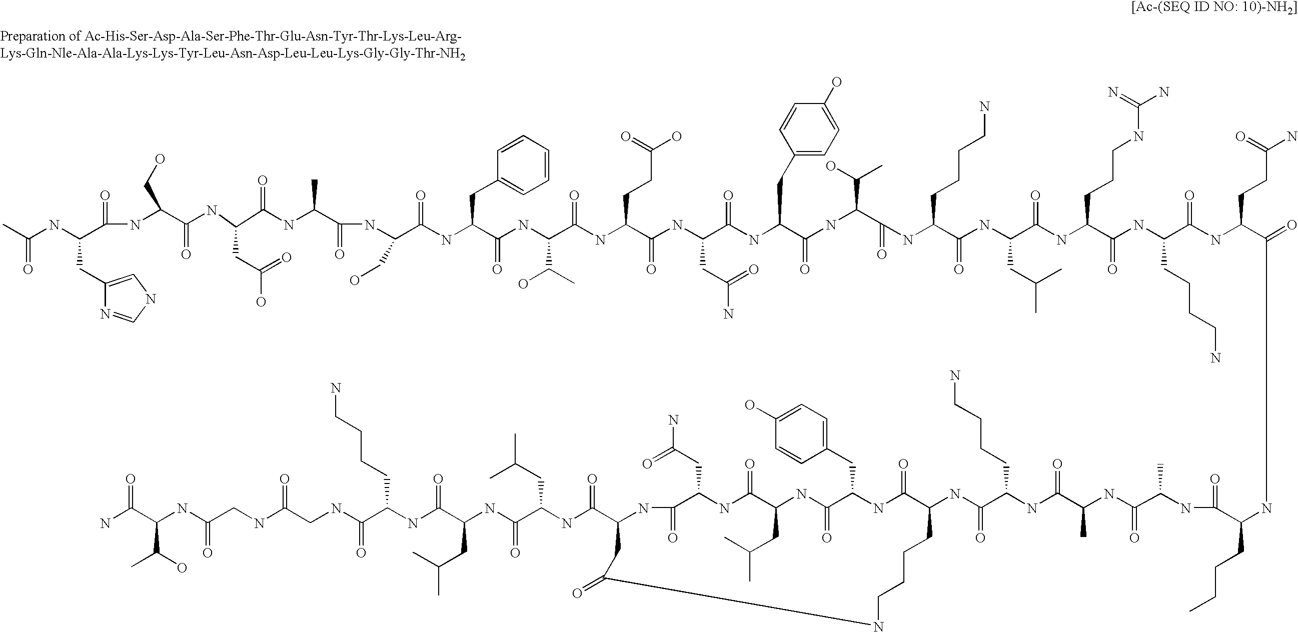

Fmoc-Rink-Linker-BHA resin (450 mg, 0.25 mmol) was subjected to solid phase synthesis and purification by following the procedure in Example 1 to yield 77 mg (7.3%) of white amorphous powder. (ES)+-LCMS m/e calcd for C₁₅₈H₂₅₃N₄₅O₄₇ 3536.00 found 3534.95.

### Example 9

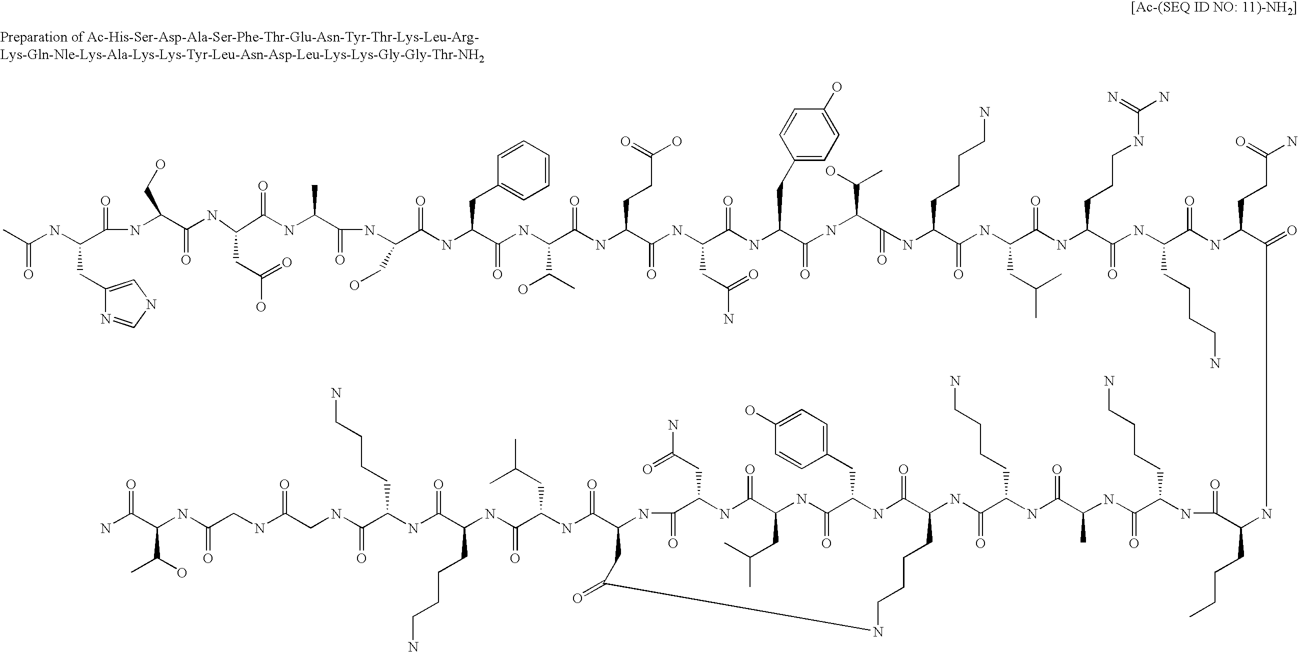

Fmoc-Rink-Linker-BHA resin (450 mg, 0.25 mmol) was subjected to solid phase synthesis and purification by following the procedure in Example 1 to yield 79 mg (7.5%) of white amorphous powder. (ES)+-LCMS m/e calcd for C₁₆₁H₂₆₁N₄₇O₄₇ 3608.11 found 3607.6.

### Example 10

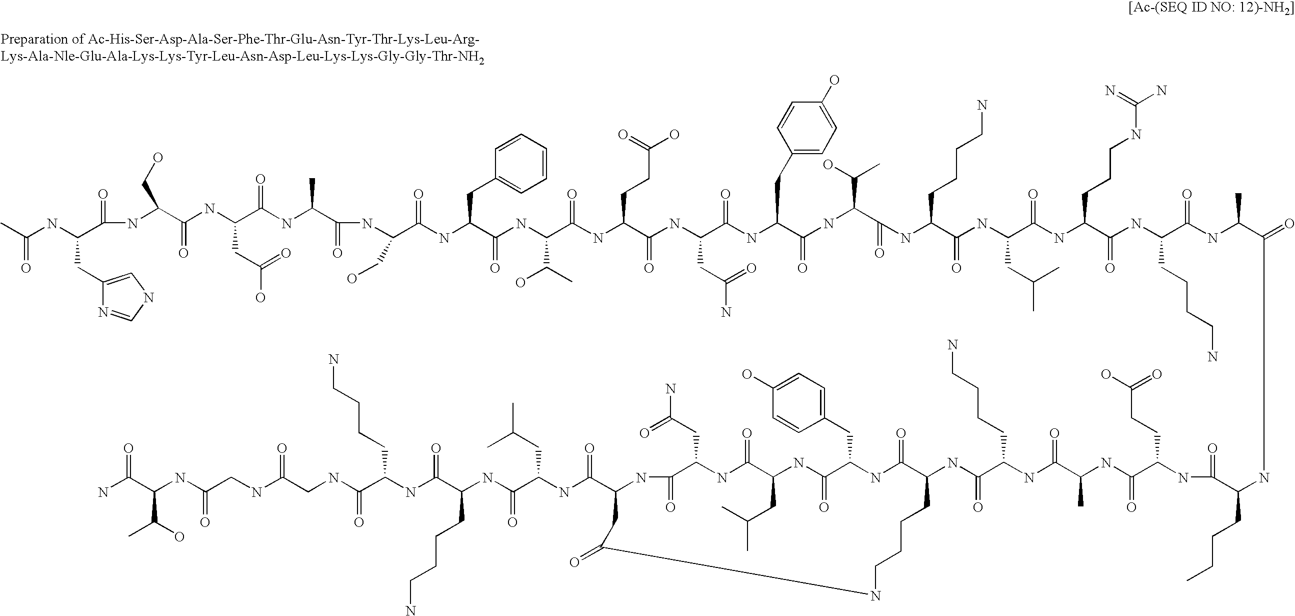

Fmoc-Rink-Linker-BHA resin (450 mg, 0.25 mmol) was subjected to solid phase synthesis and purification by following the procedure in Example 1 to yield 65 mg (6%) of white amorphous powder. (ES)+-LCMS m/e calcd for C₁₅₈H₂₅₃N₄₅O₄₈ 3552.00 found 3551.2.

### Example 11

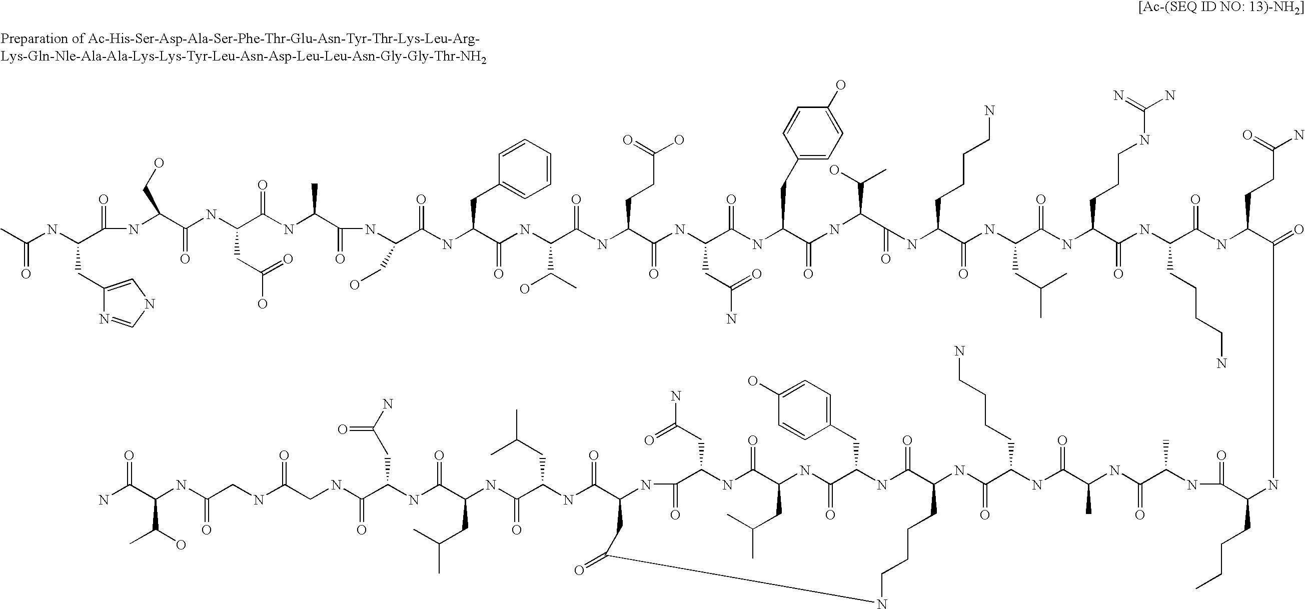

Fmoc-Rink-Linker-BHA resin (450 mg, 0.25 mmol) was subjected to solid phase synthesis and purification by following the procedure in Example 1 to yield 109 mg (10.6%) of white amorphous powder. (ES)+-LCMS m/e calcd for C₁₅₆H₂₄₇N₄₅O₄₈ 3521.93 found 3520.5.

### Example 12

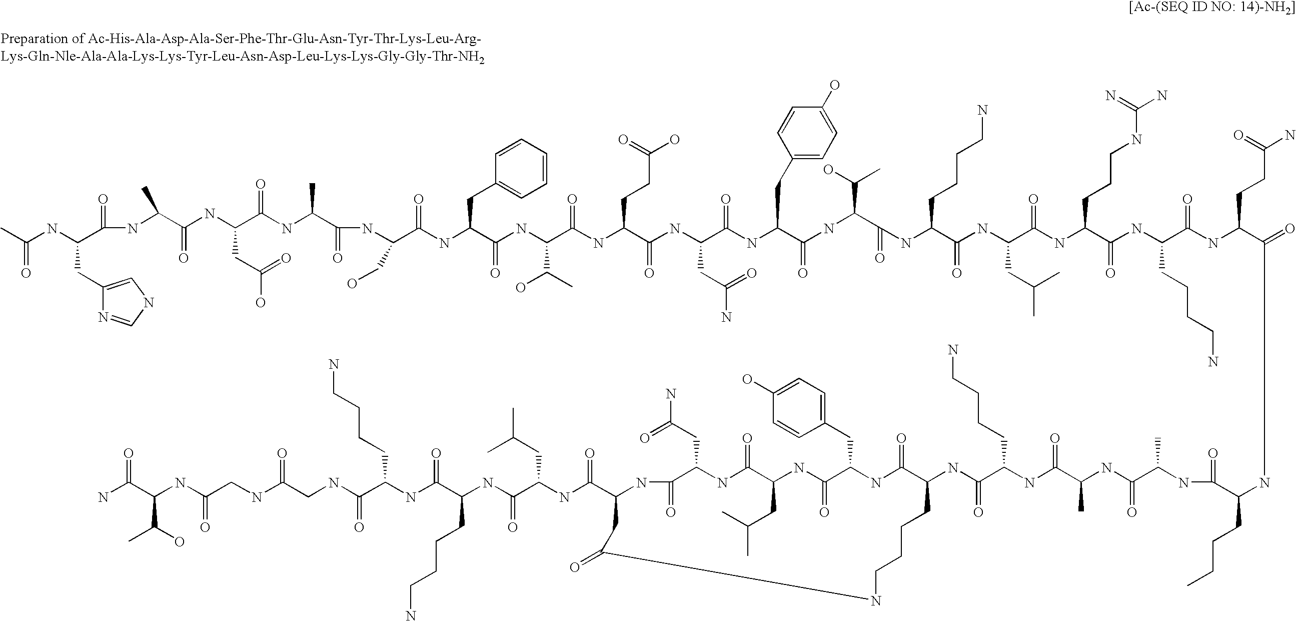

Fmoc-Rink-Linker-BHA resin (450 mg, 0.25 mmol) was subjected to solid phase synthesis and purification by following the procedure in Example 1 to yield 20 mg (1.8%) of white amorphous powder. (ES)+-LCMS m/e calcd for C₁₅₈H₂₅₄N₄₆O₄₆ 3535.02 found 3533.4.

### Example 13

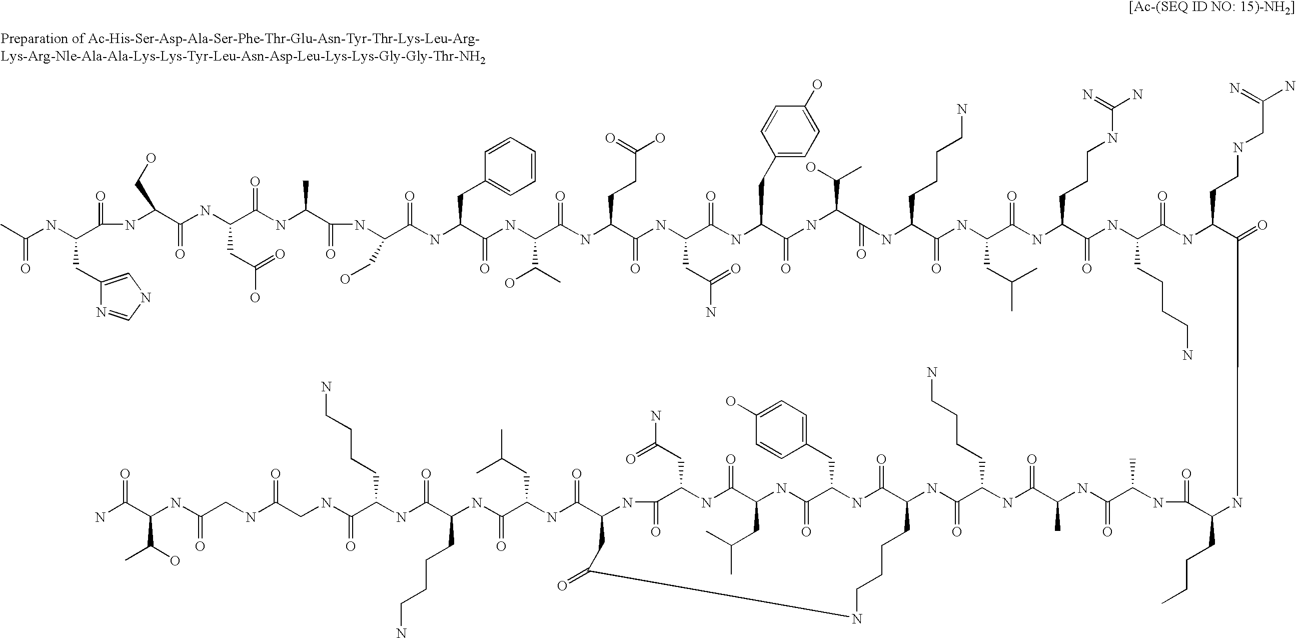

Fmoc-Rink-Linker-BHA resin (450 mg, 0.25 mmol) was subjected to solid phase synthesis and purification by following the procedure in Example 1 to yield 60 mg (5.3%) of white amorphous powder. (ES)+-LCMS m/e calcd for C₁₅₉H₂₅₈N₄₈O₄₆ 3579.08 found 3577.8.

### Example 14

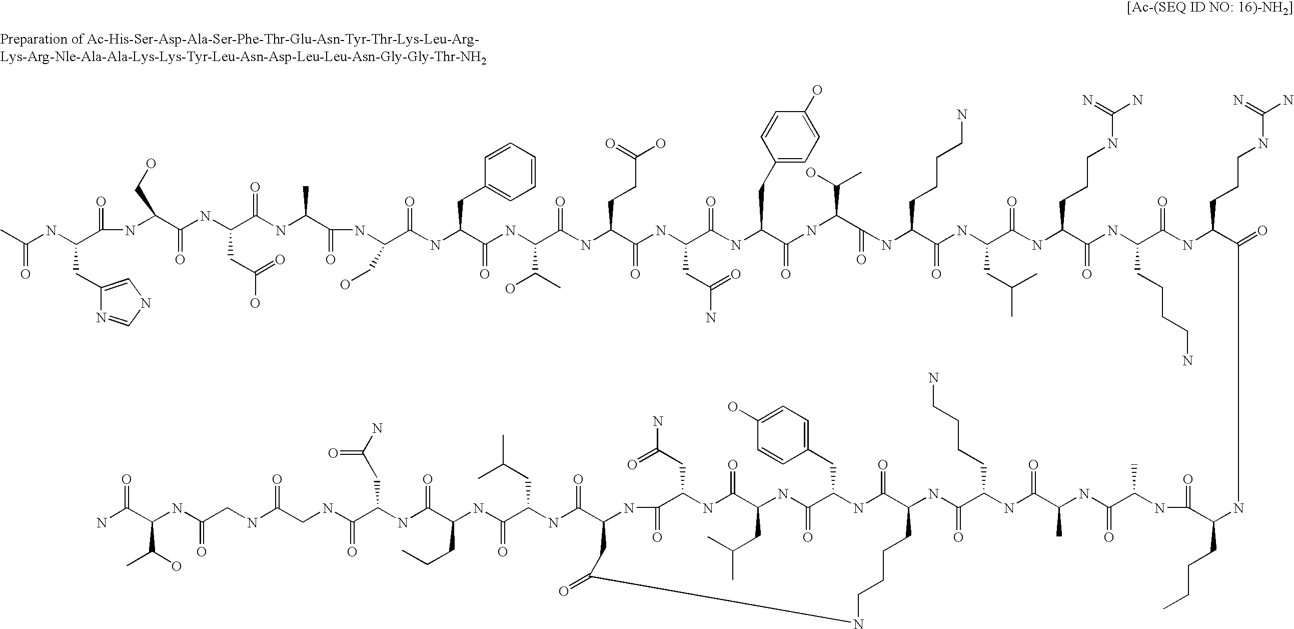

Fmoc-Rink-Linker-BHA resin (450 mg, 0.25 mmol) was subjected to solid phase synthesis and purification by following the procedure in Example 1 to yield 40 mg (3.7%) of white amorphous powder. (ES)+-LCMS m/e calcd for C₁₋₅₇H₂₅₁N₄₇O₄₇ 3549.99 found 3549.2.

### Example 15

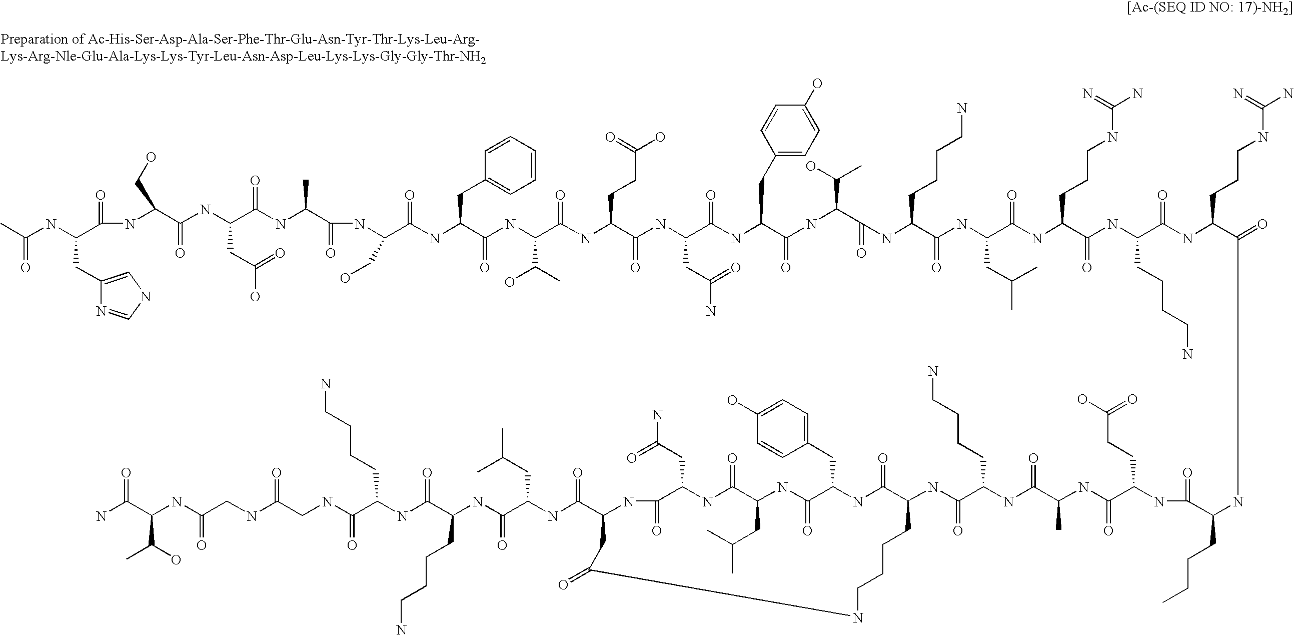

Fmoc-Rink-Linker-BHA resin (450 mg, 0.25 mmol) was subjected to solid phase synthesis and purification by following the procedure in Example 1 to yield 36 mg (3.6%) of white amorphous powder. (ES)+-LCMS m/e calcd for C₁₆₁H₂₆₀N₄₈O₄₈ 3637.11 found 3636.4.

### Example 16

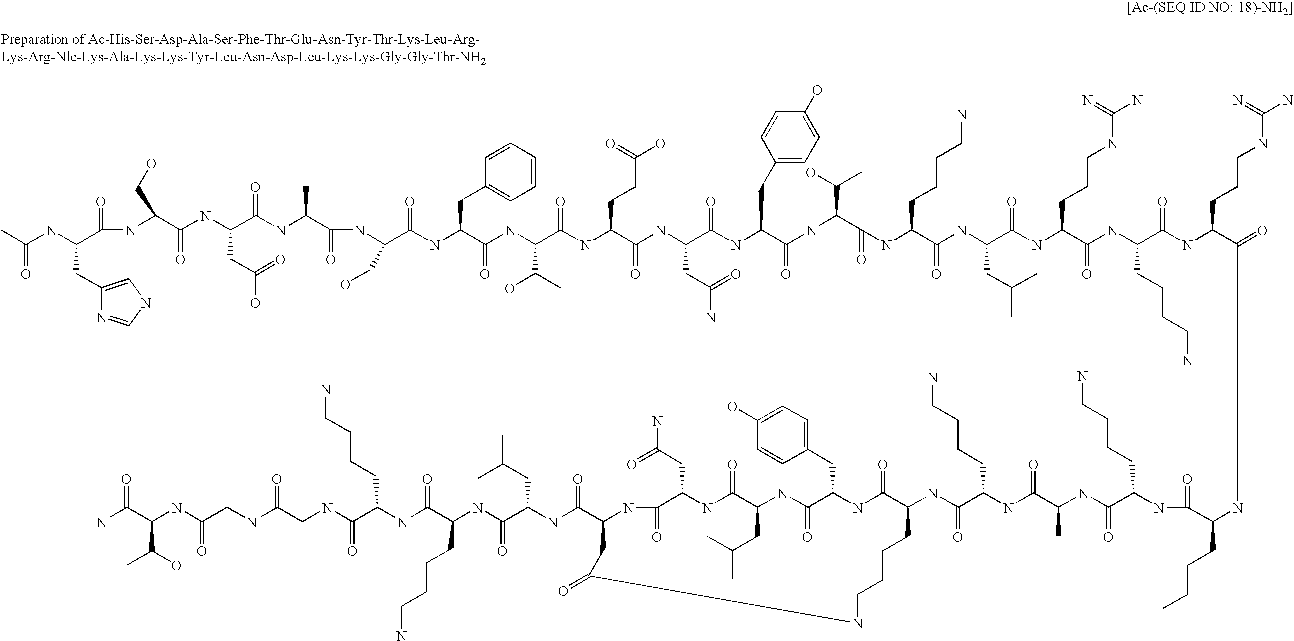

Fmoc-Rink-Linker-BHA resin (450 mg, 0.25 mmol) was subjected to solid phase synthesis and purification by following the procedure in Example 1 to yield 51 mg (4.4%) of white amorphous powder. (ES)+-LCMS m/e calcd for C₁₆₂H₂₆₅N₄₉O₄₆ 3636.17 found 3634.8.

### Example 17

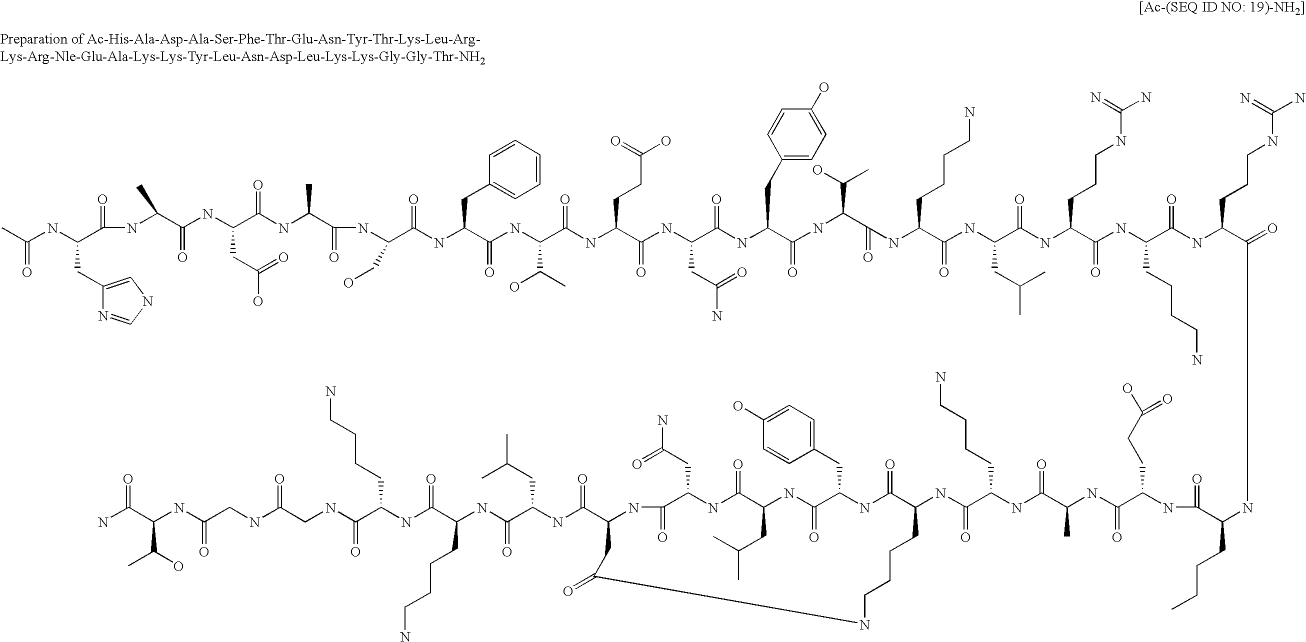

Fmoc-Rink-Linker-BHA resin (450 mg, 0.25 mmol) was subjected to solid phase synthesis and purification by following the procedure in Example 1 to yield 27 mg (2.7%) of white amorphous powder. (ES)+-LCMS m/e calcd for C₁₆₁H₂₆₀N₄₈O₄₇ 3621.11 found 3620.4.

### Example 18

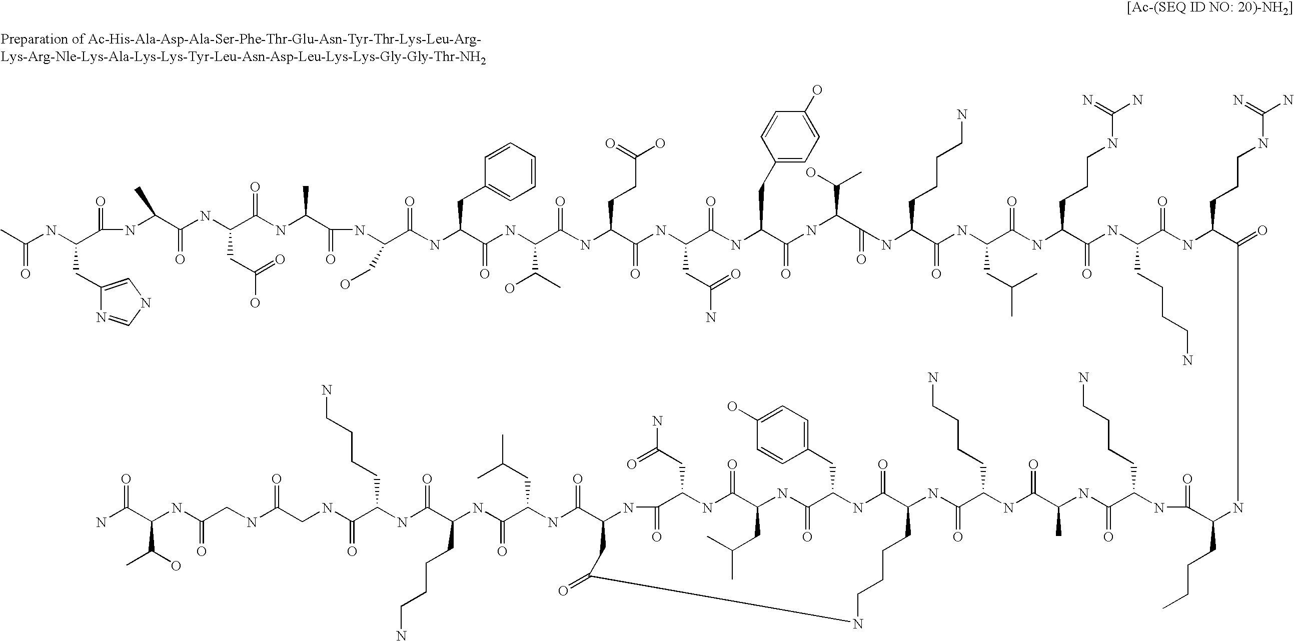

Fmoc-Rink-Linker-BHA resin (450 mg, 0.25 mmol) was subjected to solid phase synthesis and purification by following the procedure in Example 1 to yield 53.5 mg (4.6%) of white amorphous powder. (ES)+-LCMS m/e calcd for C₁₆₂H₂₆₅N₄₉O₄₅ 3620.17 found 3618.8.

### Example 19

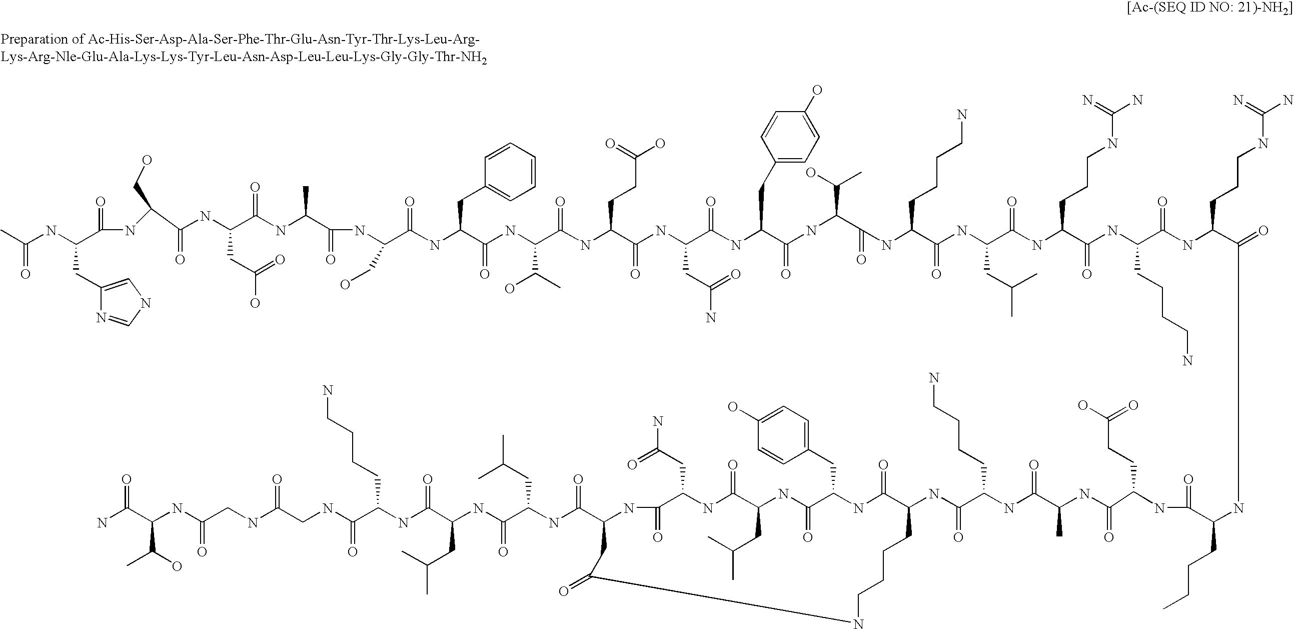

Fmoc-Rink-Linker-BHA resin (450 mg, 0.25 mmol) was subjected to solid phase synthesis and purification by following the procedure in Example 1 to yield 33 mg (3.3%) of white amorphous powder. (ES)+-LCMS m/e calcd for C₁₆₁H₂₅₉N₄₇O₄₈ 3622.10 found 3620.8.

### Example 20

### Preparation of Ac-His-Ser-Asp-Ala-MeVal-Phe-Thr-Glu-Asn-Tyr-thr-Lys-Leu-Arg-Lys-Gln-Nle-Ala-Ala-Lys-Lys-Tyr-Leu-Asn-Asp-Leu-Leu-Lys-Gly-Gly-Thr-NH₂

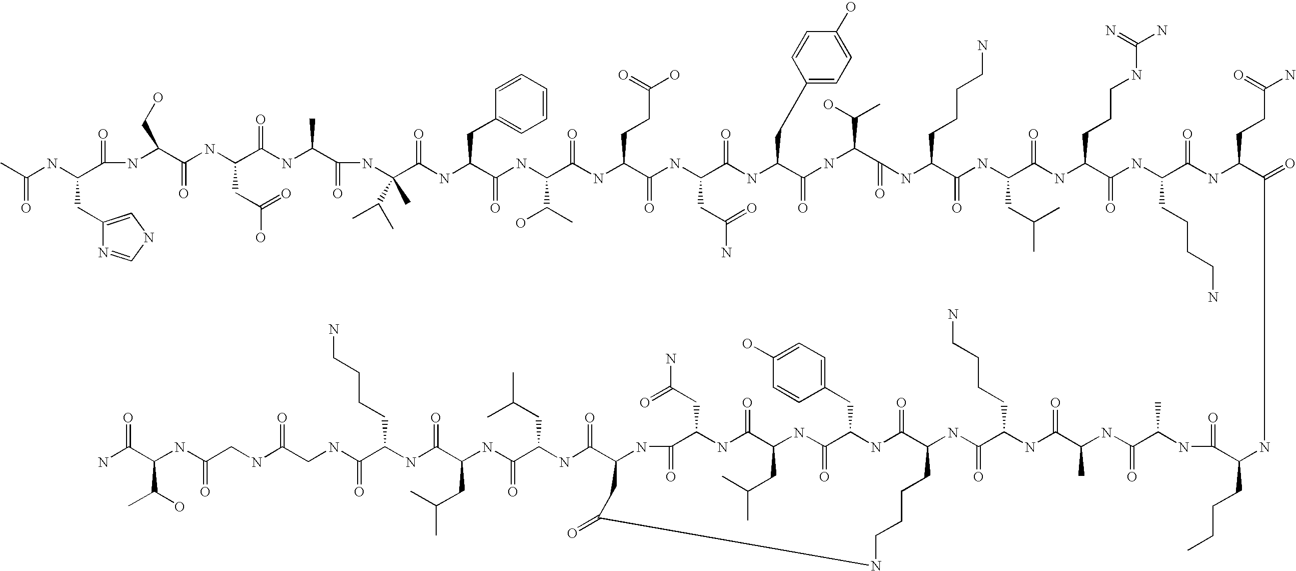

Fmoc-Rink-Linker-BHA resin (450 mg, 0.25 mmol) was subjected to solid phase synthesis and purification by following the procedure in Example 1 to yield 55 mg (5.2%) of white amorphous powder. (ES)+-LCMS m/e calcd for C₁₆₁H₂₅₉N₄₅O₄₆ 3562.09 found 3561.09.

### Example 21

### Preparation of Ac-His-Ala-Asp-Ala-MeVal-Phe-Thr-Glu-Asn-Tyr-Thr-Lys-Leu-Arg-Lys-Gln-Nle-Ala-Ala-Lys-Lys-Tyr-Leu-Asn-Asp-Leu-Lys-Lys-Gly-Gly-Thr-NH₂

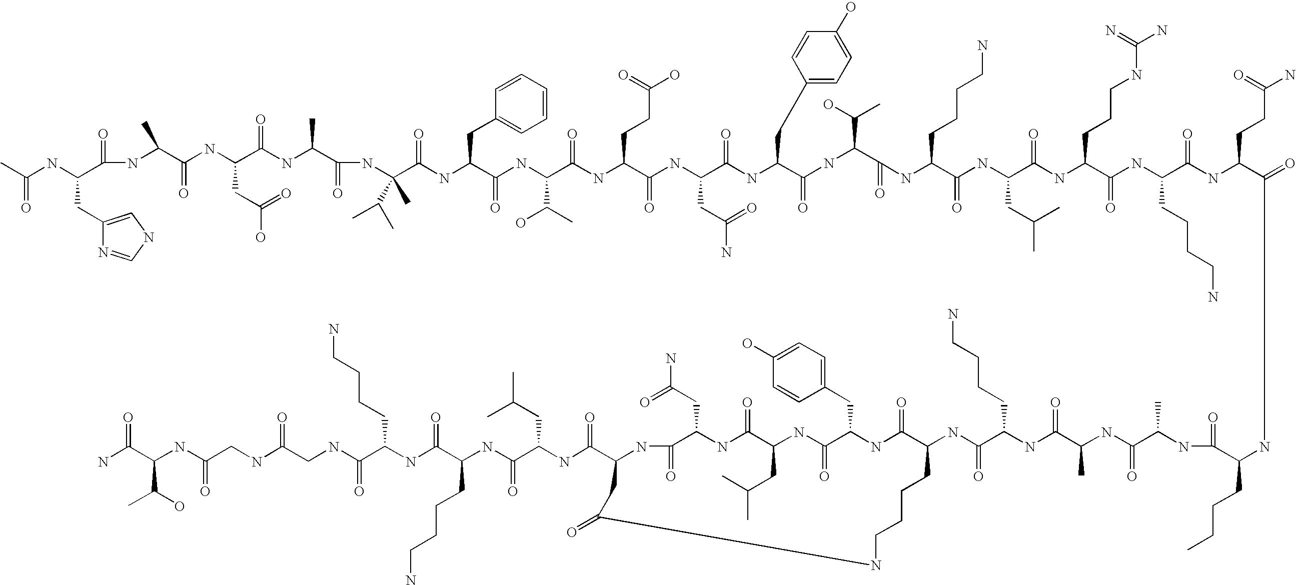

Fmoc-Rink-Linker-BHA resin (450 mg, 0.25 mmol) was subjected to solid phase synthesis and purification by following the procedure in Example 1 to yield 49 mg (4.5%) of white amorphous powder. (ES)+-LCMS m/e calcd for C₁₆₁H₂₆₀N₄₆O₄₅ 3561.10 found 3560.0.

### Example 22

### Preparation of Ac-His-Ser-Asp-Ala-MeVal-Phe-Thr-Glu-Asn-Tyr-Thr-Lys-Leu-Arg-Lys-Arg-Nle-Ala-Ala-Lys-Lys-Tyr-Leu-Asn-Asp-Leu-Lys-Lys-Gly-Gly-Thr-NH₂

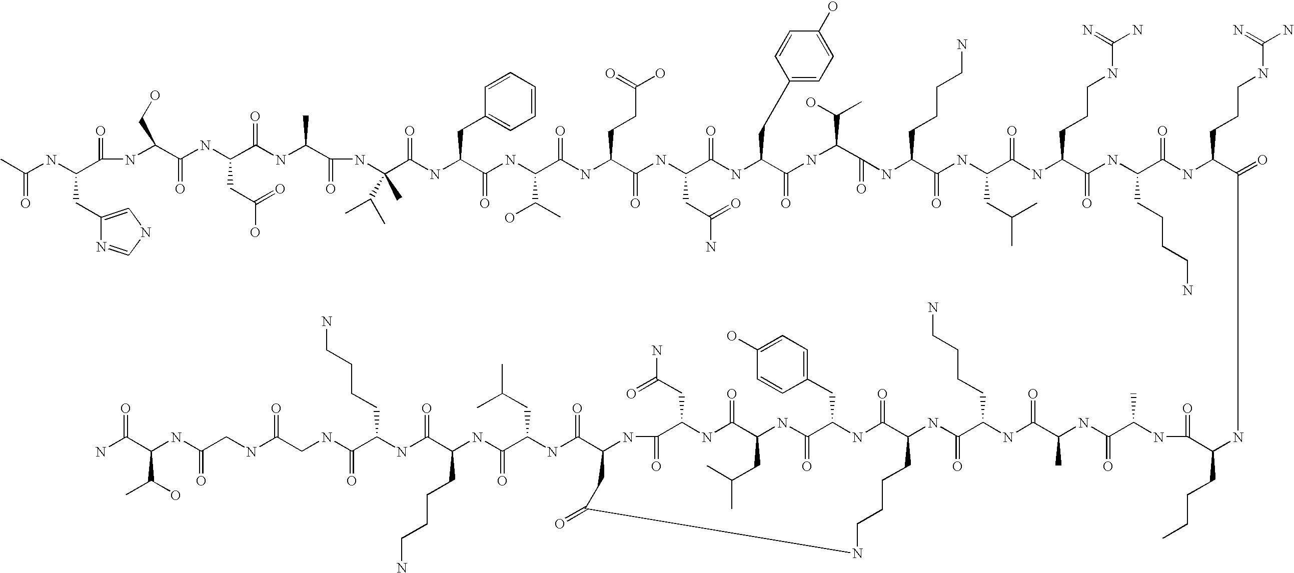

Fmoc-Rink-Linker-BHA resin (450 mg, 0.25 mmol) was subjected to solid phase synthesis and purification by following the procedure in Example 1 to yield 13.8 mg (1.2%) of white amorphous powder. (ES)+-LCMS m/e calcd for C₁₆₂H₂₆₄N₄₈O₄₅ 3605.16 found 3604.0.

### Example 23

### Preparation of Ac-His-Ala-Asp-Ala-MeVal-Phe-Thr-Glu-Asn-Tyr-Thr-Lys-Leu-Arg-Lys-Gln-Nle-Ala-Ala-Lys-Lys-Tyr-Leu-Asn-Asp-Leu-Leu-Lys-Gly-Gly-Thr-NH₂

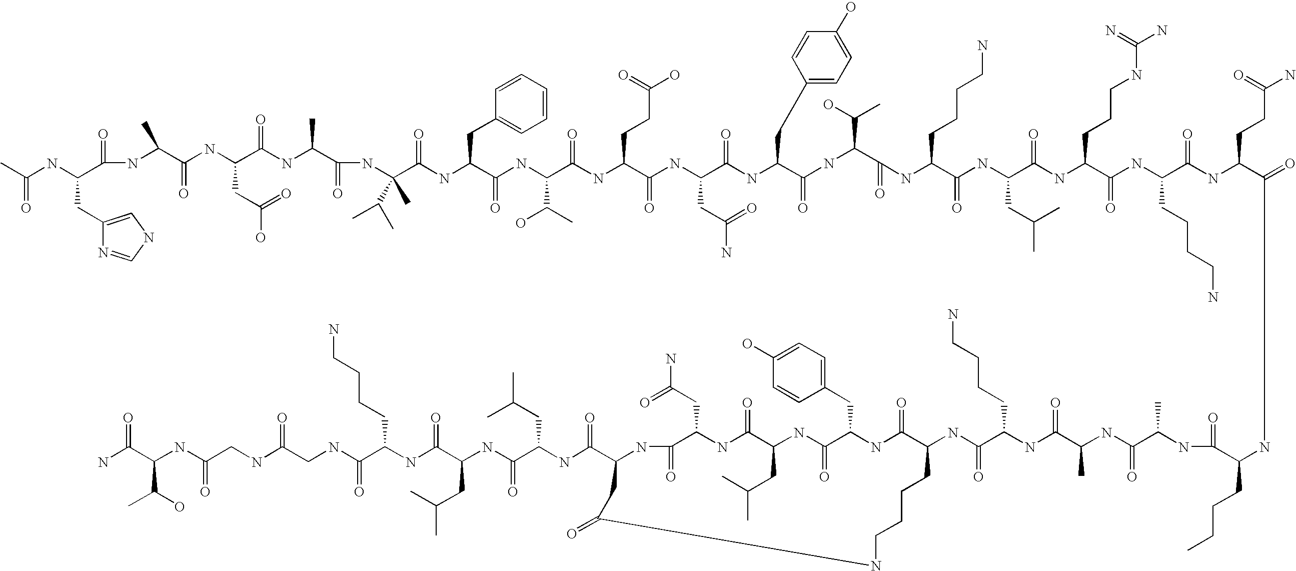

Fmoc-Rink-Linker-BHA resin (450 mg, 0.25 mmol) was subjected to solid phase synthesis and purification by following the procedure in Example 1 to yield 30.2 mg (2.8%) of white amorphous powder. (ES)+-LCMS m/e calcd for C₁₆₁H₂₅₉N₄₇O₄₅ 3546.09 found 3544.8.

### Example 24

### Sup-Ti cAMP Agonist Assay

The human T-lymphoid cell line Sup-Ti, which expresses the VPAC2 receptor, was obtained from the American Type Culture Collection (ATCC, CRL-1942) and maintained in growth medium at densities between 0.2 and 2×10⁶ cells/ml in a 37° C. CO₂ incubator. The growth medium was RPMI 1640 (Invitrogen) supplemented with 25 mM HEPES buffer and 10% fetal bovine serum (Gemini Bioproducts).

To evaluate VPAC2 agonist compound activity, cells in log-phase growth were washed once with growth medium at room temperature and plated into 96-well plates at a density of 4×10⁴ cells per well in 150 uL of growth medium. Fifty uL of the compounds to be tested, prepared at appropriate concentrations in growth medium, were then added to designated wells. After 5 min at room temperature, the cells were lysed by adding 25 uL of lysis reagent 1A (cAMP Biotrak EIA system, Amersham Biosciences, RPN225) to each well. The 96-well plates were kept at room temperature for 10 min with shaking and then stored at 4° C. until analysis for cAMP (within 2 hr).

Cyclic AMP levels were determined in 100 uL of each lysate using the cAMP Biotrak Enzymeimmunoassay (EIA) kit according to the manufacture's instructions (Amersham Biosciences, RPN225). The activity of each VPAC2 agonist compound (EC₅₀ value) was estimated by fitting the 7-concentration dose response data to a sigmoidal dose-response equation provided by the GraphPad Prism program (GraphPad Software, Inc.).

TABLE 1

| Compound in Example | Sup-T1 cAMP EC50 (nM) |
|---|---|
| 1 | 38 |
| 2 | 69.7 |
| 3 | 98 |
| 4 | 85 |
| 5 | 1206 |
| 6 | 2.35 |
| 7 | 632 |
| 8 | 11.4 |
| 9 | 1200 |
| 10 | 447 |
| 11 | 16.8 |
| 12 | 7.4 |
| 13 | 17.6 |
| 14 | 94.1 |
| 15 | 116.7 |
| 16 | 1030 |
| 17 | 23.3 |
| 18 | 276 |
| 19 | 68.4 |
| 20 | 9.45 |
| 21 | 4.75 |
| 22 | 10.54 |
| 23 | 4.07 |

### Example 25

### Peptide Stability to Neutrophil Elastase

The proteolytic stabilities of peptide analogs were established with reversed phase high pressure liquid chromatography (RP HPLC) electrospray ionization mass spectrometry (ESI MS). Peptide analogs were incubated with human neutrophile elastase and the quantity of undigested analogs was determined by ESI MS at appropriate time points. Multiple peptide analogs could be included in one experiment as long as they could be differentiated by HPLC retention time and/or by molecular weight. Ac-His Ac-His-Ser-Asp-Ala-Val-Phe-Thr-Glu-Asn-Tyr-Thr-Lys-Leu-Arg-Lys-Gln-Nle-Ala-Ala-Lys-Lys-Tyr-Leu-Asn-Asp-Leu-Lys-Lys-Gly-Gly-Thr-NH₂ was used in all experiments as a control and as a reference standard. The simultaneous use of multiple peptide analogs together with a reference standard allowed for compensation for variations in the proteolytic fidelity of the enzyme over the multiple experiments. Integrated ion currents obtained for the individual undigested peptide were used for quantitation. For calculation of halftime first-order kinetic behavior was assumed and all calculations were normalized to the halftime of the reference standard.

Peptide stock solutions were prepared in water to a concentration of 2.5 mg/mL. Unless in use, all stock solutions were kept at −20° C. In order to determine the relative peptide content in the prepared stock solutions reversed phase HPLC was done with an aliquot and the observed UV absorbance was compared with a comparable aliquot from the reference standard. Concentrations of the peptide analogs were adjusted accordingly. In order to do the proteolytic digestion, peptides were dissolved in phosphate buffered saline (PBS) to a concentration of 0.1 mg/mL. As many as six different peptide analogs were mixed into one 50 μL reaction volume. The reference standard was added to all experiments as a reference and internal standard. Elastase (Human Neutrophil, Calbiochem, Cat # 324681) was added from an elastase stock solution to a concentration of 1 to 2 μg/mL. Different amounts of the enzyme were chosen to compensate for the differences in the proteolytic stabilities of the peptide analogs. Previously, a stock solution of elastase was prepared in water at a concentration of 1 mg/mL. Small aliquots of the enzyme stock solution were kept at −20° C. to better maintain the enzyme activity by limiting the number of thaw and freeze cycles.

The digestion was done at ambient temperature in an autosampler tube within the autosampler of the HPLC system (Agilent 1100 Series). For a time course, 5 μL aliquots were injected in 70 minute intervals onto the reversed phase HPLC column (Phenomenex, Luna C18, 3μ, 100 ↑, 150×2.00 mm). For the starting time point an aliquot was injected just prior to the addition of the proteolytic enzyme. A total of eight time points could be recorded from one experiment, including the starting point. Peptides were separated on the reversed phase column with a 50 minute gradient of 5% to 30% organic phase. The aqueous phase was 0.05% (v/v) of trifluoroacetic acid in water and the organic one was 0.045% (v/v) of trifluoroacetic acid in acetonitrile. Absorbances were recorded at 214 and 280 nm respectively. All of the column effluent was introduced into the turbo V source of the electrospray ionization mass spectrometer (ABI 4000 QTrap LC/MS/MS System). Mass spectra were acquired in Q3MS mode in a mass range to include all triply charged ions of the non degraded peptide analogs. Care was taken to assure that peptide analogs could clearly be differentiated either by the chromatographic retention time or by the difference in molecular weight. Relative quantities of the respective undigested peptide analog were calculated from the integrated total ion current. A window of 2.5 Da was chosen and the manufacturer's software was used to integrate the individual ion currents. The overall halftime of an individual peptide analog was calculated by assuming first-order kinetic behavior and was normalized with respect to the halftime of the reference standard.

TABLE 2

| Compound in Example | Relative elastase stability |
|---|---|
| 1 | 3.9 |
| 2 | 5.2 |
| 3 | 4.5 |
| 4 | 3.9 |
| 5 | 4.9 |
| 6 | 6 |
| 7 | 16.4 |
| 8 | 3.5 |
| 9 | 12.0 |
| 10 | 7.4 |
| 11 | 2.4 |
| 12 | 5.2 |
| 13 | 1.7 |
| 14 | 4.5 |
| 15 | 4.8 |
| 16 | 4.4 |
| 17 | 4.6 |
| 18 | 5.1 |
| 19 | 3.3 |
| 20 | 3.4 |
| 21 | 5.7 |
| 22 | 1.8 |
| 23 | 3.6 |

### Example 26

### Effect of Compounds on LPS-Induced Lung Inflammation in Male C57BL/6 Mice

Aerosol LPS:

C57bl/6 mice are pretreated with vehicle or drug prior to an aerosol expose to lipopolysacchride (LPS, 500 μg/ml in sterile saline) for 15-30 minutes. The aerosol is generated by a Pari Ultra neb jet nebulizer, the outlet of which is connected to a small clear plastic chamber [H×W×D, 10.7×25.7×11 cm (4×10×4.5 in)] containing the animals. Bronchoalveolar lavage (BAL) is performed 24 hr later to determine the intensity of cell inflammation. BAL procedure is performed as described below.

Intranasal Administration of LPS:

Mice are pretreated with vehicle or drug prior to an intranasal administration of lipopolysacchride (0.05-0.3 mg/kg in sterile saline; 50 μl total volume, 25 μl/nostril). Intranasal administration is performed by presenting small droplets of the dosing solution at the nostril using a 25-50 μl eppendorff pipet. BAL is performed 3 to 24 h post LPS challenged as described above to determine the intensity of cell inflammation.

Bronchoalveolar Lavage:

24 h following LPS exposure, animals are anesthetized with pentobarbital (80-100 mg/kg, i.p.), ketamine/xyzaline (80-120 mg/kg/2-4 mg/kg, i.p.) or urethane (1.5-2.4 g/kg, i.p.); and through a small midline neck incision (15-20 mm), the trachea is exposed and cannulated with 20-gauge tubing adapter. Lungs are lavaged with 2×1 ml sterile Hank's balanced salt solution without Ca++ and Mg++(HBSS). Lavage fluid is recovered after 30 sec by gentle aspiration and pooled for each animal. Samples are then centrifuged at 2000 rpm for 10 minutes at 5° C. Supernatant is aspirated, and red blood cells are lysed from the resulting pellet with 0.5 ml distilled water for 30 sec before restoring osmolarity to the remaining cells by the addition of 5 ml of HBSS. Samples are recentrifuged at 2000 rpm for 10 minutes at 5° C. and supernatant aspirated. The resulting pellet is resuspended in 1 ml of HBSS. Total cell number is determined by Trypan Blue (Sigma Chemical, St. Louis, Mo.) exclusion from an aliquot of cell suspension using a hemocytometer or coulter counter. For differential cell counts, an aliquot of the cell suspension is centrifuged in a Cytospin (5 min, 1300 rpm; Shandon Southern Instruments, Sewickley, Pa.) and the slides fixed and stained with a modified Wright's stain (Hema 3 stain kit, Fisher Scientific). Standard morphological criteria is used in classifying at least 300 cells under light microscopy. Data in Table 3 is expressed as BAL cells×10⁴/animal for neutrophils and total cells, or percent inhibition of the LPS induced BAL fluid neutrophilia response.

TABLE 3

| | | Inhibition of LPS-induced neutrophilia |
|---|---|---|
| Compound in Example | Dose | (+ 10-30%, ++ >30%) |
| 1 | 0.1% | + |
| 2 | 0.1% | + |
| 6 | 0.01% | ++ |
| 7 | 0.01% | ++ |
| 8 | 0.1% | ++ |
| 9 | 0.01% | + |
| 10 | 0.01% | ++ |
| 11 | 0.01% | ++ |
| 12 | 0.01% | + |
| 13 | 0.01% | ++ |
| 15 | 0.01% | + |
| 17 | 0.01% | + |
| 19 | 0.01% | + |

### Example 27

### Effect of Compounds on Methacholine-Induced Bronchospasm in Mice

Respiratory function is measured in conscious, freely moving mice using whole body plethysmographs (WBP) from BUXCO Electronics, Inc. (Troy, N.Y.). WBP chambers allow animals to move freely within the chamber while respiratory function is measured. Eight chambers are used simultaneously so that eight mice can be measured at the same time. Each WBP chamber is connected to a bias flow regulator to supply a smooth, constant flow of fresh air during testing. A transducer attached to each chamber detects pressure changes that occur as the animal breathes. Pressure signals are amplified by a MAX II Strain Gauge preamplifier and analyzed by the Biosystem XA software supplied with the system (BUXCO Electronics, Inc.). Pressure changes within each chamber are calibrated prior to testing by injecting exactly 1 ml of air through the injection port and adjusting the computer signal accordingly. Mice are placed in the WBP chambers and allowed to acclimate for 10 minutes prior to testing. Testing is conducted by letting the animals move and breathe freely for 15 minutes while the following parameters are measured: Tidal Volume (ml), Respiratory Rate (breaths per minutes), Minute Volume (tidal volume multiplied by respiratory rate, ml/min), Inspiratory Time (sec), Expiratory Time (sec), Peak Inspiratory Flow (ml/sec), and Peak Expiratory Flow (ml/sec). Raw data for each of the parameters listed above are captured in the software database and averaged once per minute to give a total of 15 data points per parameter. The average of the 15 data points is reported. Accumulated Volume (ml) is a cumulative value (not averaged) and represents the sum of all tidal volumes for the 15-minute test session. The protocol is customized to include measurements before, during, and after a spasmogen challenge to determine Penh. Dose-response effects of a particular spasmogen (i.e. methacholine (MCh), acetylcholine, etc.) are obtained by giving nebulized aerosol (30-60 sec exposure) at approximately 5-10 min intervals. Mice (balb/c) are treated with vehicle (2% DMSO in H₂O) or drug dissolved in 4 ml vehicle for 20 minutes by aerosol, as described above, prior to spasmogen challenge. Penh is determined at 5, 30 and 60 minutes post-challenge. Data are reported as percent inhibition of Penh relative to vehicle.

TABLE 4

| | Inhibition of Penh at 5 |
|---|---|
| | minutes post-challenge |
| Compound in Example | (+ >50%, ++ <50%) |
| 6 | ++ |
| 12 | + |
| 18 | + |
| 19 | + |

1. A cyclic vasoactive intestinal peptide analog of the formula 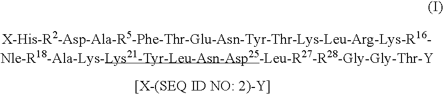
wherein:
X is a hydrogen of the N-terminal amino of Histidine which may be optionally replaced by a hydrolyzable amino protecting group,
Y is the hydroxy of the C-terminal carboxy of Threonine which may be optionally replaced by a hydrolyzable carboxy protecting group,
Lys²¹ has a side-chain to side-chain covalent linkage to Asp²⁵,
R² is Ser or Ala,
R⁵ is Thr, Ser, Asp, Gln, Pro or CαMeVal,
R¹⁶ is Gln, Ala, or Arg,
R¹⁸ is Ala, Lys or Glu,
R²⁷ is Lys or Leu except that R²⁷ must be Lys when R⁵ is CαMeVal and R¹⁶ is Arg,
R²⁸ is Lys or Asn,
or a pharmaceutically acceptable salt thereof.

2. The compound of claim 1 wherein R⁵ is Ser or CαMeVal.

3. The compound of claim 2 wherein R⁵ is CαMeVal.

4. The compound of claim 2 wherein R²⁷ is Lys.

5. The compound of claim 1 wherein X is hydrogen or Ac and Y is hydrogen or NH₂.

6. A compound selected from the group consisting of
(SEQ ID NO:3)
His-Ser-Asp-Ala-Thr-Phe-Thr-Glu-Asn-Tyr-Thr-Lys-
Leu-Arg-Lys-Gln-Nle-Ala-Ala-Lys-Lys-Tyr-Leu-Asn-
Asp-Leu-Lys-Lys-Gly-Gly-Thr,
(SEQ ID NO:4)
His-Ser-Asp-Ala-Ser-Phe-Thr-Glu-Asn-Tyr-Thr-Lys-
Leu-Arg-Lys-Gln-Nle-Ala-Ala-Lys-Lys-Tyr-Leu-Asn-
Asp-Leu-Lys-Lys-Gly-Gly-Thr,
(SEQ ID NO:5)
His-Ser-Asp-Ala-Asp-Phe-Thr-Glu-Asn-Tyr-Thr-Lys-
Leu-Arg-Lys-Gln-Nle-Ala-Ala-Lys-Lys-Tyr-Leu-Asn-
Asp-Leu-Lys-Lys-Gly-Gly-Thr,
(SEQ ID NO:6)
His-Ser-Asp-Ala-Gln-Phe-Thr-Glu-Asn-Tyr-Thr-Lys-
Leu-Arg-Lys-Gln-Nle-Ala-Ala-Lys-Lys-Tyr-Leu-Asn-
Asp-Leu-Lys-Lys-Gly-Gly-Thr,
(SEQ ID NO:7)
His-Ser-Asp-Ala-Pro-Phe-Thr-Glu-Asn-Tyr-Thr-Lys-
Leu-Arg-Lys-Gln-Nle-Ala-Ala-Lys-Lys-Tyr-Leu-Asn-
Asp-Leu-Lys-Lys-Gly-Gly-Thr,
(SEQ ID NO:8)
His-Ser-Asp-Ala-MeVal-Phe-Thr-Glu-Asn-Tyr-Thr-Lys-
Leu-Arg-Lys-Gln-Nle-Ala-Ala-Lys-Lys-Tyr-Leu-Asn-
Asp-Leu-Lys-Lys-Gly-Gly-Thr,
(SEQ ID NO:9)
His-Ser-Asp-Ala-Ser-Phe-Thr-Glu-Asn-Tyr-Thr-Lys-
Leu-Arg-Lys-Gln-Nle-Glu-Ala-Lys-Lys-Tyr-Leu-Asn-
Asp-Leu-Lys-Lys-Gly-Gly-Thr,
(SEQ ID NO:10)
His-Ser-Asp-Ala-Ser-Phe-Thr-Glu-Asn-Tyr-Thr-Lys-
Leu-Arg-Lys-Gln-Nle-Ala-Ala-Lys-Lys-Tyr-Leu-Asn-
Asp-Leu-Lys-Lys-Gly-Gly-Thr,
(SEQ ID NO:11)
His-Ser-Asp-Ala-Ser-Phe-Thr-Glu-Asn-Tyr-Thr-Lys-
Leu-Arg-Lys-Gln-Nle-Lys-Ala-Lys-Lys-Tyr-Leu-Asn-
Asp-Leu-Lys-Lys-Gly-Gly-Thr,
(SEQ ID NO:12)
His-Ser-Asp-Ala-Ser-Phe-Thr-Glu-Asn-Tyr-Thr-Lys-
Leu-Arg-Lys-Ala-Nle-Glu-Ala-Lys-Lys-Tyr-Leu-Asn-
Asp-Leu-Lys-Lys-Gly-Gly-Thr,
(SEQ ID NO:13)
His-Ser-Asp-Ala-Ser-Phe-Thr-Glu-Asn-Tyr-Thr-Lys-
Leu-Arg-Lys-Gln-Nle-Ala-Ala-Lys-Lys-Tyr-Leu-Asn-
Asp-Leu-Leu-Asn-Gly-Gly-Thr,
(SEQ ID NO:14)
His-Ala-Asp-Ala-Ser-Phe-Thr-Glu-Asn-Tyr-Thr-Lys-
Leu-Arg-Lys-Gln-Nle-Ala-Ala-Lys-Lys-Tyr-Leu-Asn-
Asp-Leu-Lys-Lys-Gly-Gly-Thr,
(SEQ ID NO:15)
His-Ser-Asp-Ala-Ser-Phe-Thr-Glu-Asn-Tyr-Thr-Lys-
Leu-Arg-Lys-Arg-Nle-Ala-Ala-Lys-Lys-Tyr-Leu-Asn-
Asp-Leu-Lys-Lys-Gly-Gly-Thr,
(SEQ ID NO:16)
His-Ser-Asp-Ala-Ser-Phe-Thr-Glu-Asn-Tyr-Thr-Lys-
Leu-Arg-Lys-Arg-Nle-Ala-Ala-Lys-Lys-Tyr-Leu-Asn-
Asp-Leu-Leu-Asn-Gly-Gly-Thr,
(SEQ ID NO:17)
His-Ser-Asp-Ala-Ser-Phe-Thr-Glu-Asn-Tyr-Thr-Lys-
Leu-Arg-Lys-Arg-Nle-Glu-Ala-Lys-Lys-Tyr-Leu-Asn-
Asp-Leu-Lys-Lys-Gly-Gly-Thr,
(SEQ ID NO:18)
His-Ser-Asp-Ala-Ser-Phe-Thr-Glu-Asn-Tyr-Thr-Lys-
Leu-Arg-Lys-Arg-Nle-Lys-Ala-Lys-Lys-Tyr-Leu-Asn-
Asp-Leu-Lys-Lys-Gly-Gly-Thr,
(SEQ ID NO:19)
His-Ala-Asp-Ala-Ser-Phe-Thr-Glu-Asn-Tyr-Thr-Lys-
Leu-Arg-Lys-Arg-Nle-Glu-Ala-Lys-Lys-Tyr-Leu-Asn-
Asp-Leu-Lys-Lys-Gly-Gly-Thr,
(SEQ ID NO:20)
His-Ala-Asp-Ala-Ser-Phe-Thr-Glu-Asn-Tyr-Thr-Lys-
Leu-Arg-Lys-Arg-Nle-Lys-Ala-Lys-Lys-Tyr-Leu-Asn-
Asp-Leu-Lys-Lys-Gly-Gly-Thr,
(SEQ ID NO:21)
His-Ser-Asp-Ala-Ser-Phe-Thr-Glu-Asn-Tyr-Thr-Lys-
Leu-Arg-Lys-Arg-Nle-Glu-Ala-Lys-Lys-Tyr-Leu-Asn-
Asp-Leu-Leu-Lys-Gly-Gly-Thr,
(SEQ ID NO:22)
His-Ser-Asp-Ala-MeVal-Phe-Thr-Glu-Asn-Tyr-Thr-Lys-
Leu-Arg-Lys-Gln-Nle-Ala-Ala-Lys-Lys-Tyr-Leu-Asn-
Asp-Leu-Leu-Lys-Gly-Gly-Thr,
(SEQ ID NO:23)
His-Ala-Asp-Ala-MeVal-Phe-Thr-Glu-Asn-Tyr-Thr-Lys-
Leu-Arg-Lys-Gln-Nle-Ala-Ala-Lys-Lys-Tyr-Leu-Asn-
Asp-Leu-Lys-Lys-Gly-Gly-Thr,
(SEQ ID NO:24)
His-Ser-Asp-Ala-MeVal-Phe-Thr-Glu-Asn-Tyr-Thr-Lys-
Leu-Arg-Lys-Arg-Nle-Ala-Ala-Lys-Lys-Tyr-Leu-Asn-
Asp-Leu-Lys-Lys-Gly-Gly-Thr,
and
(SEQ ID NO:25)
His-Ala-Asp-Ala-MeVal-Phe-Thr-Glu-Asn-Tyr-Thr-Lys-
Leu-Arg-Lys-Gln-Nle-Ala-Ala-Lys-Lys-Tyr-Leu-Asn-
Asp-Leu-Leu-Lys-Gly-Gly-Thr.

7. The compound of claim 1 which is X-(SEQ ID NO: 3)-Y.

8. The compound of claim 1 which is X-(SEQ ID NO: 4)-Y.

9. The compound of claim 1 which is X-(SEQ ID NO: 5)-Y.

10. The compound of claim 1 which is X-(SEQ ID NO: 6)-Y.

11. The compound of claim 1 which is X-(SEQ ID NO: 7)-Y.

12. The compound of claim 1 which is X-(SEQ ID NO: 8)-Y.

13. The compound of claim 1 which is X-(SEQ ID NO: 9)-Y.

14. The compound of claim 1 which is X-(SEQ ID NO: 10)-Y.

15. The compound of claim 1 which is X-(SEQ ID NO: 11)-Y.

16. The compound of claim 1 which is X-(SEQ ID NO: 12)-Y.

17. The compound of claim 1 which is X-(SEQ ID NO: 13)-Y.

18. The compound of claim 1 which is X-(SEQ ID NO: 14)-Y.

19. The compound of claim 1 which is X-(SEQ ID NO: 15)-Y.

20. The compound of claim 1 which is X-(SEQ ID NO: 16)-Y.

21. The compound of claim 1 which is X-(SEQ ID NO: 17)-Y.

22. The compound of claim 1 which is X-(SEQ ID NO: 18)-Y.

23. The compound of claim 1 which is X-(SEQ ID NO: 19)-Y.

24. The compound of claim 1 which is X-(SEQ ID NO: 20)-Y.

25. The compound of claim 1 which is X-(SEQ ID NO: 21)-Y.

26. The compound of claim 1 which is X-(SEQ ID NO: 22)-Y.

27. The compound of claim 1 which is X-(SEQ ID NO: 23)-Y.

28. The compound of claim 1 which is X-(SEQ ID NO: 24)-Y.

29. The compound of claim 1 which is X-(SEQ ID NO: 25)-Y.

30. A pharmaceutical composition for inhalation administration comprising a compound of claim 1 and at least one pharmaceutically acceptable carrier or excipient in solution or micronized dry powder form wherein the compound is present in a pharmacologically effective concentration for pulmonary delivery of said composition.

31. The composition of claim 30 wherein the concentration of the compound is sufficient to deliver from about 1 μg/kg to about 50 μg/kg of the compound in a single inhaled dose.

32. A method for treating pulmonary obstructive disorders comprising administering by inhalation an effective amount of the composition of claim 31 to a person suffering from such disorder.

33. The method of claim 32 wherein the lung disorder is COPD.

34. The method of claim 32 wherein the amount of the compound administered is from about 1 μg/kg/day to about 50 μg/kg/day.
